(19) United States
(12) Patent Application Publication
(10) Pub. No.: US 2023/0392002 A1
(43) Pub. Date: Dec. 7, 2023

(54) METHOD FOR EVALUATING COMPATIBILITY OF THERMOSETTING RESIN COMPOSITION, THERMOSETTING RESIN COMPOSITION, PREPREG, RESIN FILM, LAMINATED PLATE, MULTILAYER PRINTED WIRING BOARD, AND SEMICONDUCTOR PACKAGE

(71) Applicant: Resonac Corporation, Tokyo (JP)

(72) Inventors: Shuichi ISHIBASHI, Tokyo (JP); Daisuke FUJIMOTO, Tokyo (JP); Tomohiko KOTAKE, Tokyo (JP)

(21) Appl. No.: 18/257,146

(22) PCT Filed: Dec. 10, 2021
(86) PCT No.: PCT/JP2021/045494

§ 371 Date: Jun. 13, 2023

(30) Foreign Application Priority Data
Dec. 14, 2020 (JP) 2020-206800

(51) Int. Cl. C08L 35/00; C08K 3/36; C08J 5/24; C08J 5/18; G01N 23/2251; G01N 33/44; H01L 23/498

(52) U.S. Cl. CPC C08L 35/00; C08K 3/36; C08J 5/249; C08J 5/18; G01N 23/2251; G01N 33/44; H01L 23/49894; H01L 23/49822; C08L 2205/035; C08L 2203/20; C08J 2335/00; C08J 2471/12; C08J 2447/00; C08J 2453/02

(57) ABSTRACT

A method for evaluating the compatibility of a thermosetting resin composition containing at least two kinds of resins and an inorganic filler, the method including the following steps 1A and 2A: Step 1A: a step of obtaining a reflected electronic image of the cross section of a cured product of the thermosetting resin composition using a scanning electron microscope at an observation magnification of 50 to 250 times; and Step 2A: a step in which, in the reflected electronic image, a phase-separated resin region is referred to as a separation part and the remaining region is referred to as a non-separation part, and the image is binarized such that the separation part has one value and the non-separation part has the other value, and the area ratio of the region of the non-separation part of the resultant binarized image to the total region of the binarized image (area of the region of the non-separation part×100/area of the total region of the binarized image) is calculated as the area ratio Rw of the non-separation part.

### TECHNICAL FIELD

The present embodiment relates to a method for evaluating the compatibility of a thermosetting resin composition, and to a thermosetting resin composition, a prepreg, a resin film, a laminated plate, a multilayer printed wiring board, and a semiconductor package.

### BACKGROUND ART

In a mobile communication device typified by a mobile phone, its base station apparatus, a network infrastructure device such as a server and a router, and an electronic device such as a large-sized computer, speeding up and increasing in the capacity of a signal to be used is progressing year by year. With that, a substrate material of a printed wiring board to be mounted on such an electronic device is required to have dielectric characteristics capable of reducing the transmission loss of a high-frequency signal (hereinafter this may be referred to as “high-frequency characteristics”), i.e., a low relative dielectric constant and a low dielectric tangent.

In recent years, in addition to the electronic devices described above, also in the field of automobile and traffic system-related ITS and in the field of in-room short-range communication, a new system that handles high-frequency radio signals has been put into practical use or planned for practical use. Therefore, it is expected that the need for a substrate material excellent in high-frequency characteristics also increases for printed wiring boards for use in these fields in the future.

Heretofore, thermoplastic polymers excellent in high frequency characteristics have been used in printed wiring boards requiring low transmission loss. As the thermoplastic polymer, for example, polymers having no polar group in the molecule such as polyphenylene ether and polybutadiene are effective for reduction in dielectric tangent. However, these thermoplastic polymers have a low compatibility with other resins having a polar group, and when they are formed into a resin composition, problems such as separation from other resins have occurred. It is desirable to suppress the separation of the resins because it may cause a decrease in workability, a decrease in homogeneity of products and a decrease in physical properties owing to it.

PTL 1 discloses a thermosetting resin composition containing a specific polyphenylene ether and a polyfunctional vinyl aromatic copolymer. In the technique of PTL 1, the compatibility of the polyfunctional vinyl aromatic copolymer with an epoxy resin is evaluated by dissolving a polyfunctional vinyl aromatic copolymer, an epoxy resin and a phenol resin in a solvent followed by visually confirming the transparency of the dissolved sample.

### CITATION LIST

### Patent Literature

PTL 1: JP 2018-168347A

### SUMMARY OF INVENTION

### Technical Problem

However, in the evaluation method described in PTL 1, the transparency can change by the compatibility between the resin and the solvent of a dispersion medium, and therefore it is impossible to know how the different resins can be compatible with each other in the cured product obtained finally. In addition, in the case where there is a difference in the compatibility to such an extent that it can be visually discriminated, it is possible to determine the superiority or inferiority of the compatibility, but in the case where there is a difference in the compatibility to such an extent that it cannot be visually discriminated, there is a problem that it is impossible to determine the superiority or inferiority of the compatibility. Under the situation that development of a resin composition having further more improved dielectric characteristics and heat resistance is desired, there is an increasing need to design materials while more strictly evaluating the compatibility between resins.

In consideration of the current situation as above, an object of the present embodiment is to provide a method for evaluating the compatibility of a thermosetting resin composition, a thermosetting resin composition having improved compatibility, as well as a prepreg, a resin film, a laminated plate, a multilayer printed wiring board and a semiconductor package using the thermosetting resin composition.

### Solution to Problem

The present inventors have made investigations for solving the above-mentioned problems and, as a result, have found that the problems can be solved by the present embodiment as follows.

Specifically, the present embodiment relates to the following [1] to [9].

[1] A method for evaluating a compatibility of a thermosetting resin composition containing at least two kinds of resins and an inorganic filler, the method including the following steps 1A and 2A:
Step 1A: a step of obtaining a reflected electronic image of a cross section of a cured product of the thermosetting resin composition using a scanning electron microscope at an observation magnification of 50 to 250 times; and
Step 2A: a step in which, in the reflected electronic image, a phase-separated resin region is referred to as a separation part and a remaining region is referred to as a non-separation part, and the image is binarized such that the separation part has one value and the non-separation part has the other value, and an area ratio of a region of the non-separation part of the resultant binarized image to a total region of the binarized image (the area of the region of the non-separation part×100/area of the total region of the binarized image) is calculated as the area ratio R_{w} of the non-separation part.
   [2] A method for evaluating a compatibility of a thermosetting resin composition containing at least two kinds of resins and an inorganic filler, the method including the following steps 1B and 2B:
Step 1B: a step of obtaining a reflected electronic image of a cross section of a cured product of the thermosetting resin composition using a scanning electron microscope; and
Step 2B: a step in which, in the reflected electronic image, a phase-separated resin region is referred to as a separation part and an average domain size D_{L} of the separation part is determined.
   [3] The compatibility evaluation method of a thermosetting resin composition according to the above [2], wherein the step 1B is a step of obtaining the reflected electronic image of the cross section of the cured product of the thermosetting resin composition using the scanning electron microscope at an observation magnification of 50 to 200 times.
   [4] A thermosetting resin composition including at least two kinds of resins and an inorganic filler, wherein:
the area ratio R_{w} of the non-separation part obtained with a scanning electron microscope under the condition of an observation magnification of 100 times or 200 times in the compatibility evaluation method of the above [1] is 50% or more, and
in the compatibility evaluation method of the above [2], the condition of the observation magnification of the scanning electron microscope is 65 times, and the average domain size D_{L} of the separation part, as obtained by averaging the domain size values of the domains having a size ranging from the 2nd to the 6th among the domains having a larger domain size counted from the larger domain size among the domains in the separation part observed in at least three fields of view, is 120 μm or less.
   [5] A prepreg including the thermosetting resin composition of the above [4].
   [6] A resin film including the thermosetting resin composition of the above [4].
   [7] A laminated plate including the prepreg of the above [5] and a metal foil.
   [8] A multilayer printed wiring board including at least one selected from the group consisting of the prepreg of the above [5], the resin film of the above [6] and the laminated plate of the above [7].
   [9] A semiconductor package formed using the printed wiring board of the above [8].

### Advantageous Effects of Invention

According to the present embodiment, there can be provided a method for evaluating the compatibility of a thermosetting resin composition, a thermosetting resin composition having improved compatibility, as well as a prepreg, a resin film, a laminated plate, a multilayer printed wiring board and a semiconductor package using the thermosetting resin composition.

### BRIEF DESCRIPTION OF DRAWING

FIG. 1 is one example of a reflected electronic image obtained in the compatibility evaluation of the present embodiment.

FIG. 2 is a schematic view showing a measurement method for the domain size of a separation part.

FIG. 3 is another schematic view showing a measurement method for the domain size of a separation part.

### DESCRIPTION OF EMBODIMENTS

In the present specification, a numerical value range expressed using “to” denotes a range including numerical values before and after “to” as a minimum value and a maximum value, respectively.

A lower limit and an upper limit in a numerical range described in the present specification can be arbitrarily combined with a lower limit or an upper limit in any other numerical range.

In a numerical range described in the present specification, an upper limit or a lower limit in the numerical range can be replaced with a value shown in Examples.

Unless otherwise specifically indicated, the components and the materials exemplified in the present specification can be used alone or in combination of two or more thereof.

Unless otherwise specifically indicated, in the present specification, regarding the content of each component in the thermosetting resin composition, in the case where plural kinds of substances corresponding to the component exist in the thermosetting resin composition, the content means a total content of the plural kinds of substances existing in the thermosetting resin composition.

Embodiments in which the matters described in the present specification are combined arbitrarily are also included in the present embodiment.

The mechanism of action described in the present specification is inferred and does not limit the mechanism of the effect of the thermosetting resin composition according to the present embodiment.

The term “compatible” in the present specification means that the resins are miscible in a nano or micro unit, or in appearance, even though they are not necessarily compatible in a molecule unit.

The number-average molecular weight in the present specification means a value measured in terms of polystyrene in gel permeation chromatography (GPC), and specifically can be measured according to the method described in Examples.

In the following description, the thermosetting resin composition can be abbreviated simply as “resin composition”. [Compatibility Evaluation Method of First Mode]

The compatibility evaluation method of the first mode of the present embodiment is a method for evaluating the compatibility of a thermosetting resin composition containing at least two kinds of resins and an inorganic filler, and the method includes the following steps 1A and 2A:

Step 1A: a step of obtaining a reflected electronic image of the cross section of a cured product of the thermosetting resin composition using a scanning electron microscope at an observation magnification of 50 to 250 times; and

Step 2A: a step in which, in the reflected electronic image, a phase-separated resin region is referred to as a separation part and the remaining region is referred to as a non-separation part, and the image is binarized such that the separation part has one value and the non-separation part has the other value, and the area ratio of the region of the non-separation part of the resultant binarized image to the total region of the binarized image (area of the region of the non-separation part×100/area of the total region of the binarized image) is calculated as the area ratio R_{w} of the non-separation part.

According to the compatibility evaluation method of the first mode of the present embodiment, the compatibility of a resin composition can be quantified, and can be evaluated more strictly than heretofore-known compatibility evaluation methods.

Hereinunder, the steps of the compatibility evaluation method of the first mode of the present embodiment are described in detail.

### <Step 1A>

The step 1A is a step of obtaining a reflected electronic image of the cross section of a cured product of a thermosetting resin composition containing at least two kinds of resins and an inorganic filler, using a scanning electron microscope (SEM) at an observation magnification of 50 to 250 times.

The resin composition that is an object to be measured in the compatibility evaluation method of the present embodiment is not specifically limited so far as it is a resin composition containing at least two kinds of resins and an inorganic filler, and for example, the resin composition of the present embodiment to be mentioned below can be the object to be measured.

The curing condition for the resin composition is not specifically limited, and the resin composition can be cured under the condition suitable for the resin composition of an object to be measured. For example, in the case where the resin composition of the present embodiment to be mentioned below is an object to be measured, the condition can be as described in Examples. Specifically, the resin composition is dried under heat at 170° C. for 5 minutes to be in a B-stage condition, and then hot press-molded under the condition of a temperature of 230° C., a pressure of 2.0 MPa and a time of 120 minutes to give a cured product.

The method of forming the cross section of a cured product of the resin composition is not specifically limited, and any heretofore-known method is employable. For example, employable is a method of forming a cross section of a cured product using a precision cutting machine, an ion milling machine or an ultrasonic cutting machine. In forming a cross section, from the viewpoint of workability, a cured product of the resin composition can be in a state embedded in an embedding resin. As needed, the cross section formed can be polished.

After the cross section of a cured product is formed, preferably, the cross section is plated by vapor deposition with platinum or the like, for better SEM observation.

The object for SEM observation obtained in the above step is referred to as “test piece”.

Next, the cross section of the test piece obtained in the above is observed with SEM.

The SEM observation in the compatibility evaluation method of the present embodiment is carried out in a reflection electron mode especially for increasing the contrast between the inorganic filler and the resin components.

The acceleration voltage in SEM observation can be appropriately adjusted depending on the object to be measured, and can be adjusted, for example, in a range of 0.5 to 20 kV.

The SEM observation magnification in the step 1A falls within a range of 50 to 250 times, and is preferably 60 to 230 times, more preferably 80 to 210 times. By controlling the observation magnification to fall within the range, it is possible to suppress the variation in measurement due to the field of view and to increase the reproducibility of the calculated area ratio R_{w} of the non-separated part. In particular, the compatibility evaluation method of the first mode of the present embodiment enables observation at a relatively low magnification and therefore makes it possible to observe the phase-separated resin region and the other regions as domains seemingly having a uniform hue, and makes it easy to carry out the step 2A to be mentioned below.

The step 1A gives a reflected electronic image of the test piece.

### <Step 2A>

The step 2A is a step in which, in the reflected electronic image, a phase-separated resin region is referred to as a separation part and the remaining region is referred to as a non-separation part, the values are binarized such that the separation part has one value and the non-separation part has the other value, and the area ratio of the region of the non-separation part of the resultant binarized image to the total region of the binarized image (area of the region of the non-separation part×100/area of the total region of the binarized image) is calculated as an area ratio R_{w} of the non-separation part.

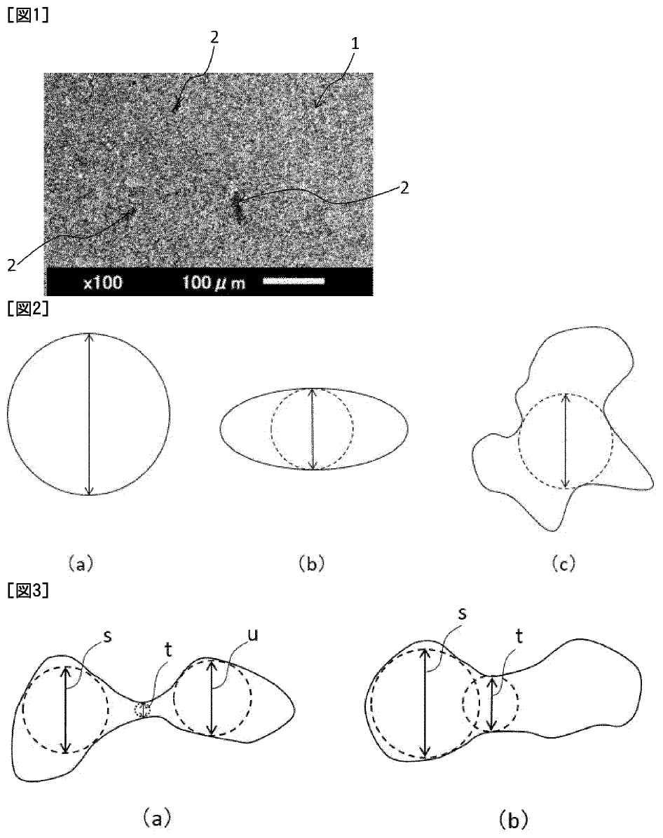

FIG. 1 shows one example of the reflected electronic image obtained in the step 1A. As shown in FIG. 1, the reflected electronic image obtained in the step 1A contains a region 1 looking relatively light and a region 2 looking relatively dark. The region looking relatively dark is a resin-rich phase having a low electron density, and corresponds to the phase-separated resin region. On the other hand, the region looking relatively light is a phase containing compatibilized resins and an inorganic filler having a high electron density.

In the present step, the phase-separated resin region is specified as “separation part”, and the other region is specified as “non-separation part”. As obvious from FIG. 1, in the reflected electronic image, the separation part can be clearly and easily specified visually.

Next, the reflected electronic image is binarized such that the separation part has one value and the non-separation part has the other value, and thereafter the area ratio of the region of the non-separation part of the resultant binarized image to the total region of the binarized image (area of the region of the non-separation part×100/area of the total region of the binarized image) is calculated as the area ratio R_{w} of the non-separation part.

Binarization in the present step is, for example, a treatment of giving a pixel value “1 (white)” to the pixels having a pixel value not lower than a predetermined threshold value, and giving a pixel value “0 (black)” to the other pixels.

Binarization can be carried out according to a known method and, for example, can be carried out using commercially-available image-editing software.

The condition for binarization can be appropriately adjusted depending on the reflected electronic image obtained in the step 1A, and the image can be binarized under the condition under which the separation part has one value and the non-separation part has the other value. Specifically, for example, in binarization using an image processing analyzer software by Nippon Roper K.K. (Image-Pro Analyzer 7.0J), the threshold value of RGB is appropriately adjusted within a range of 40 to 100 as a processing condition for binarization.

Next, the area ratio of the region of the non-separation part of the resultant binarized image to the total region of the binarized image (area of the region of the non-separation part×100/area of the total region of the binarized image) is calculated as the area ratio R_{w} of the non-separation part. For example, the area ratio R_{w} can be calculated by counting the pixel number in the total region of the binarized image and the pixel number of the values indicating the non-separation part.

In the compatibility evaluation method of the first mode, the area ratio R_{w} of the non-separation part in one field of view can be calculated, but from the viewpoint of reproducibility, it is preferable that the area ratio R_{w} of the non-separation part in plural fields of view is individually calculated and thereafter the data are averaged. The number of the fields of view to be averaged is not specifically limited, and for example, can be 2, 3, 4 or 5 fields of view, and can be appropriately selected depending on the desired accuracy.

The area ratio R_{w} of the non-separation part obtained according to the above-mentioned method can be an index of compatibility. Specifically, a larger area ratio R_{w} of the non-separation part means that the resin composition contains a smaller amount of phase-separated resin and is excellent in compatibility.

### [Compatibility Evaluation Method of Second Mode]

The compatibility evaluation method of the second mode of the present embodiment is a method for evaluating the compatibility of a thermosetting resin composition containing at least two kinds of resins and an inorganic filler, and the method includes the following steps 1B and 2B:
Step 1B: a step of obtaining a reflected electronic image of the cross section of a cured product of the thermosetting resin composition using a scanning electron microscope; and
Step 2B: a step in which, in the reflected electronic image, a phase-separated resin region is referred to as a separation part and the average domain size D_{L} of the separation part is determined.

According to the compatibility evaluation method of the second mode of the present embodiment, the compatibility of a resin composition can be quantified, and can be evaluated more strictly than heretofore-known compatibility evaluation methods.

Hereinunder, the steps of the compatibility evaluation method of the second mode of the present embodiment are described in detail.

### <Step 1B>

The step 1B is a step of obtaining a reflected electronic image of the cross section of a cured product of a thermosetting resin composition containing at least two kinds of resins and an inorganic filler, using a scanning electron microscope.

Though not specifically limited, the observation magnification of the scanning electron microscope in the step 1B is, from the viewpoint of workability and reproducibility, preferably 30 to 500 times, more preferably 40 to 200 times, even more preferably 50 to 200 times, specially preferably 50 to 100 times.

The description of the step 1B except the observation magnification is the same as that of the step 1A.

### <Step 2B>

The step 2B is a step in which, in the reflected electronic image, a phase-separated resin region is referred to as a separation part and the average domain size D_{L} of the separation part is determined.

Here, the domain size in the present step is defined as a diameter of a largest true circle that can be drawn inside the domain of the separation part.

FIG. 2 and FIG. 3 each are a schematic view showing a domain size measurement method for the separation part.

For example, as shown in FIG. 2(a), in the case where the domain is considered as a true circle, the diameter thereof corresponds to the domain size. Also for example, as shown in FIG. 2(b) or (c), in the case where the domain is considered as an ellipse or an indefinite shape, the diameter of a largest true circle that can be drawn inside the ellipse or the indefinite shape corresponds to the domain size.

The domain of a separation part can have a shape of two or more linking domains as shown in FIGS. 3(a) and (b). In that case, the diameter of a largest true circle that can be drawn inside the domain is referred to as s, and the diameter of a largest true circle that can be drawn inside the linking part is referred to as t, and when the diameter t is not more than ⅓ of the diameter s, the domain is considered as divided by the linking part. Specifically, the domain shown in FIG. 3(a) is considered to have two domains of a domain having a diameter s and a domain having a diameter u, both existing therein. On the other hand, in the domain shown in FIG. 3(b), the diameter t is more than ⅓ of the diameter s, and the domain is therefore not divided by the linking part and considered as one domain having a domain size of the diameter s.

In the step 2B, the average domain size D_{L} of the separation part is preferably one determined by averaging the domain sizes of the domains having the 2nd largest and subsequent sizes as counted from those having larger domain sizes among the domains of the separation part. With that, in production of a resin composition, the influence of a large separation part to be generated unintentionally owing to any other factor than compatibility can be excluded to thereby better the reproducibility.

The number of the 2nd and subsequent domains for calculating the average value is not specifically limited, and can be appropriately determined depending on the object to be measured. From the viewpoint of workability and reproducibility, the average domain size D_{L} of the separation part is one determined by averaging the domain sizes of the domains having sizes within a range of the 2nd or more and the 100th or less, as counted sequentially from larger domains, preferably the domains having a size of the 50th or less, more preferably the domains having a size of the 10th or less, even more preferably the domains having a size within a range of the 2nd or more and the 6th or less.

In the compatibility evaluation method of the second mode, the average domain size D_{L} can be determined by carrying out the above mentioned step for one field of view alone, but from the viewpoint of reproducibility, it is preferable that after plural fields of view are observed, the average domain size D_{L} is determined from all the domains contained in the plural fields of view. In the case where plural fields of view are observed, the number thereof is preferably at least 3 fields of view, more preferably at least 5 fields of view, even more preferably at least 6 fields of view. The upper limit of the number of the fields of view to be observed is not specifically limited, and can be, for example 20 fields of view or less, or can be 15 fields of view or less, or can be 10 fields of view or less.

The average domain size D_{L} of the separation part obtained according to the above-mentioned method can be an index of compatibility. Specifically, a smaller average domain size D_{L} of the separation part means that the resin composition is excellent in compatibility.

### [Thermosetting Resin Composition]

Next described is the thermosetting resin composition of the present embodiment.

The thermosetting resin composition of the present embodiment is a thermosetting resin composition containing at least two kinds of resins and an inorganic filler, wherein:
the area ratio R_{w} of the non-separation part obtained with a scanning electron microscope under the condition of an observation magnification of 100 times or 200 times in the compatibility evaluation method of the above-mentioned first mode is 50% or more, and
in the compatibility evaluation method of the above-mentioned second mode, the condition of the observation magnification of the scanning electron microscope is 65 times, and the average domain size D_{L} of the separation part, as obtained by averaging the domain sizes of the domains having sizes ranging from the 2nd to the 6th among the domains having larger domain sizes counted from the larger domain sizes among the domains in the separation part observed in at least three fields of view, is 120 μm or less.

The area ratio R_{w} of the non-separation pat and the average domain size D_{L} relating to the thermosetting resin composition of the present embodiment are the values measured according to the compatibility evaluation method of the first mode and the compatibility evaluation method of the second mode mentioned above, and are, more specifically, the values measured according to the methods described in Examples.

The area ratio R_{w} of the non-separation part in the resin composition of the present embodiment is, though not specifically limited, preferably 52% or more, more preferably 54% or more, even more preferably 56% or more. When the area ratio R_{w} of the non-separation part is the above-mentioned lower limit or more, the dielectric characteristics and the heat resistance tend to better.

The upper limit of the area ratio R_{w} of the non-separation part is not specifically limited, and can be 100%, but from the viewpoint of ease of production, it can be 98% or less, or can be 95% or less.

The average domain size D_{L} of the separation part in the resin composition of the present embodiment is, though not specifically limited, preferably 100 μm or less, more preferably 90 μm or less, even more preferably 85 μm or less. When the average domain size D_{L} of the separation part is the above-mentioned upper limit or less, the dielectric characteristics and the heat resistance tend to better.

The lower limit of the average domain size D_{L} of the separation part is not specifically limited, and can be 0 μm, but from the viewpoint of ease of production, it can be 10 m or more, or can be 30 μm or more. The average domain size D_{L} of the separation part being 0 μm means that the separation part is not substantially observed and the domain size cannot be measured.

The area ratio R_{w} of the non-separation part and the average domain size D_{L} of the separation part each can be adjusted to fall within the above-mentioned range, for example, by selecting the kind of the resin to be contained in the resin composition.

The resin composition of the present embodiment contains at least two kinds of resins and an inorganic filler.

The resin components are not specifically limited so far as they satisfy the above-mentioned average domain size D_{L} of the non-separation part and the above-mentioned area ratio R_{w} of the separation part, but at least two kinds, an elastomer and a thermosetting resin are preferably contained.

Examples of the elastomer include a polyether-based elastomer, a styrene-based elastomer, a conjugated diene-based elastomer, an urethane-based elastomer, a polyester-based elastomer, a polyamide-based elastomer, an acryl-based elastomer, and a silicone-based elastomer. One kind alone of elastomer or two or more kinds thereof can be used either singly or as combined. Among the above, the elastomer is, from the viewpoint of dielectric characteristics, preferably any of a polyether-based elastomer, a styrene-based elastomer and a conjugated diene-based elastomer.

Examples of the thermosetting resin include an epoxy resin, a cyanate ester compound, a maleimide compound, a bisallyl nadimide resin, a benzoxazine compound, and derivatives thereof. One kind alone of thermosetting resin or two or more kinds thereof can be used either singly or as combined. Among these, the thermosetting resin is, from the viewpoint of heat resistance, low thermal expansion property and mechanical characteristics, preferably a maleimide compound or a derivative thereof.

Among the above-mentioned resins, the resin composition of the present embodiment preferably contains a polyether-based elastomer as an elastomer and contains a maleimide compound or a derivative thereof as a thermosetting resin.

More preferably, the resin composition of the present embodiment contains a polyphenylene ether derivative having an ethylenically-unsaturated bond-containing group [hereinafter this may be referred to as “polyphenylene ether derivative (A)” or “component (A)”] as an elastomer and contains at least one selected from the group consisting of a maleimide compound having at least two N-substituted maleimide groups and a derivative thereof [hereinafter this may be referred to as “maleimide compound or a derivative thereof (B)” or “component (B)”] as a thermosetting resin.

Even more preferably, the resin composition of the present embodiment contains, as an elastomer in addition to the component (A) and the component (B), at least two selected from the group consisting of (C) a conjugated diene polymer or a modified conjugated diene polymer [hereinafter this may be referred to as “conjugated diene polymer or a modified product thereof (C)” or “component (C)”], and (D) a styrene-based thermoplastic elastomer [hereinafter this may be referred to as “styrene-based thermoplastic elastomer (D)” or “component (D)”].

Especially preferably, the resin composition of the present embodiment contains, as a curing accelerator, (E) an imidazole compound or a modified imidazole compound [hereinafter this may be referred to as “imidazole compound or a modified product thereof (E)” or “component (E)”].

Hereinunder, the components that the resin composition of the present embodiment preferably contains are described sequentially in detail.

### <Polyphenylene Ether Derivative (A)>

The component (A) has an ethylenically-unsaturated bond-containing group. Specifically, the component (A) can be said to be a polyphenylene ether introduced with an ethylenically-unsaturated bond-containing group.

In the present specification, “ethylenically-unsaturated bond” means an addition reaction-acceptable carbon-carbon double bond, and does not contain a double bond of an aromatic ring. “Ethylenically-unsaturated bond-containing group” means a substituent that contains the above-mentioned ethylenically-unsaturated bond.

One kind alone of the component (A) or two or more kinds thereof can be used either singly or as combined.

In the component (A), the position of the ethylenically-unsaturated bond-containing group is not specifically limited, and the group can be at the terminal, or can be in any other position than the terminal. In the case where the component (A) has an ethylenically-unsaturated bond-containing group at the terminal, the position of the ethylenically-unsaturated bond-containing group can be at one terminal or can be at both terminals. “Terminal” of the component (A) means not only the atom to be at the farthest end of the molecule but also the entire organic group that bonds to the ether bond existing at the farthest end of the polyphenylene ether chain from the end side. Specifically, the component (A) having an ethylenically-unsaturated bond-containing group at the terminal has the same meaning as that of the case where the organic group bonding to the ether bond existing at the farthest end of the polyphenylene ether chain from the end side has an ethylenically-unsaturated bond-containing group.

The component (A) can be a mixture of a polyphenylene ether derivative having an ethylenically-unsaturated bond-containing group at one terminal and a polyphenylene ether derivative having ethylenically-unsaturated bond-containing groups at both terminals, but preferably contains at least a polyphenylene ether derivative having an ethylenically-unsaturated bond-containing group at one terminal, and is more preferably a polyphenylene ether derivative itself having an ethylenically-unsaturated bond-containing group at one terminal.

In the case where the component (A) contains a polyphenylene ether derivative having an ethylenically-unsaturated bond-containing group at one terminal, the content of the polyphenylene ether derivative having an ethylenically-unsaturated bond-containing group at one terminal in the component (A) is preferably 30% by mass or more, more preferably 45% by mass or more, even more preferably 55% by mass or more, further more preferably 70% by mass or more, specially preferably 90% by mass or more, and is most preferably substantially 100% by mass.

Examples of the ethylenically-unsaturated bond-containing group that the component (A) has include an unsaturated aliphatic hydrocarbon group such as a vinyl group, an isopropenyl group, an allyl group, a 1-methylallyl group and a 3-butenyl group; and a hetero atom-containing substituent such as a maleimide group and a (meth)acryloyl group. Among these, from the viewpoint of dielectric characteristics, preferred are an unsaturated aliphatic hydrocarbon group and a maleimide group, more preferred are an allyl group and a maleimide group, and even more preferred is an allyl group.

In the present specification, the unsaturated aliphatic hydrocarbon group described as the ethylenically-unsaturated bond-containing group does not contain a hetero atom.

Next, the polyphenylene ether derivative having an unsaturated aliphatic hydrocarbon group as an ethylenically-unsaturated bond-containing group is described in more detail.

The number of the unsaturated aliphatic hydrocarbon groups that the component (A) has in one molecule is, though not specifically limited but from the viewpoint of dielectric characteristics, preferably 2 or more, more preferably 3 or more, even more preferably 4 or more. The upper limit of the number of the unsaturated aliphatic hydrocarbon groups that the component (A) has in one molecule is not specifically limited, and can be 8 or less, or can be 7 or less, or can be 6 or less.

The number of the unsaturated aliphatic hydrocarbon groups that the component (A) has at one terminal is, though not specifically limited but from the viewpoint of dielectric characteristics, preferably 2 or more, more preferably 3 or more, even more preferably 4 or more. The upper limit of the number of the unsaturated aliphatic hydrocarbon groups that the component (A) has at one terminal is not specifically limited, and can be 8 or less, or can be 7 or less, or can be 6 or less.

The number of the unsaturated aliphatic hydrocarbon groups that the component (A) has and the number of the unsaturated aliphatic hydrocarbon groups that the component (A) has at one terminal each are most preferably 4.

From the viewpoint of dielectric characteristics, the component (A) preferably contains a structure represented by the following general formula (a-1).

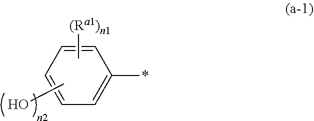

In the formula, R^{a1} is an unsaturated aliphatic hydrocarbon group having 2 to 10 carbon atoms. n1 is 1 or 2, and n2 is 0 or 1. * indicates a bonding position to the other structure.

In the above general formula (a-1), the unsaturated aliphatic hydrocarbon group having 2 to 10 carbon atoms that R^{a1} represents is, among the above-mentioned ones, from the viewpoint of dielectric characteristics, preferably an unsaturated aliphatic hydrocarbon group having 2 to 5 carbon atoms, more preferably a vinyl group, an isopropenyl group, an allyl group, a 1-methylallyl group or a 3-butenyl group, even more preferably an allyl group.

In the case where n1 is 2, plural Rai's can be the same as or different from each other.

From the viewpoint of dielectric characteristics, the component (A) is also preferably an embodiment containing a structure represented by the following general formula (a-2).

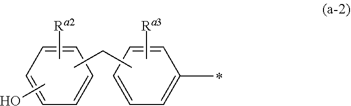

In the formula, R^{a2} and R^{a3} each independently are an unsaturated aliphatic hydrocarbon group having 2 to 10 carbon atoms. * indicates a bonding position to the other structure.

The unsaturated aliphatic hydrocarbon group having 2 to 10 carbon atoms that R^{a2} and R^{a3} in the above general formula (a-2) represent each can be the same one as R^{a1} in the above general formula (a-1), and each are preferably the same one.

More preferably from the viewpoint of dielectric characteristics, the component (A) contains a structure represented by any of the following general formulae (a-3) to (a-5), even more preferably contains a structure represented by the general formula (a-5).

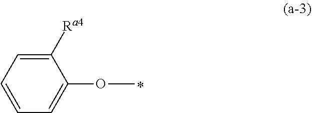

In the formula, R^{a4} are an unsaturated aliphatic hydrocarbon group having 2 to 10 carbon atoms. * indicates a bonding position to the other structure.

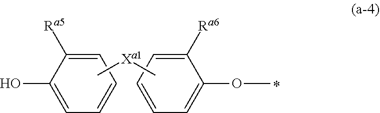

In the formula, R^{a5} and R^{a6} each independently are an unsaturated aliphatic hydrocarbon group having 2 to 10 carbon atoms. X^{a1} is a divalent aliphatic hydrocarbon group having 1 to 6 carbon atoms. * indicates a bonding position to the other structure.

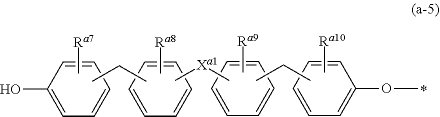

In the formula, R^{a7} to R^{a10} each independently are an unsaturated aliphatic hydrocarbon group having 2 to 10 carbon atoms. X^{a2} is a divalent organic group. * indicates a bonding position to the other structure.

The unsaturated aliphatic hydrocarbon group having 2 to 10 carbon atoms that R^{a4} to R^{a10} in the general formulae (a-3) to (a-5) represent each can be the same one as R^{a1} in the above general formula (a-1), and preferred ones thereof are also the same as those of the latter.

Examples of the divalent aliphatic hydrocarbon group having 1 to 6 carbon atoms that X^{a1} in the general formula (a-4) represents include an alkylene group having 1 to 6 carbon atoms such as a methylene group, an ethylene group and a trimethylene group; and an alkylidene group having 2 to 6 carbon atoms such as an isopropylidene group. Among these, preferred are a methylene group and an isopropylidene group, and more preferred is an isopropylidene group.

Examples of the divalent organic group that X^{a2} in the general formula (a-5) represents include an aliphatic hydrocarbon group optionally having a hetero atom in a part thereof, an alicyclic hydrocarbon group optionally having a hetero atom in a part thereof, an aromatic hydrocarbon group optionally having a hetero atom in a part thereof, and a group of an arbitrary combination of these groups.

Examples of the hetero atom include an oxygen atom, a nitrogen atom, and a sulfur atom.

The divalent organic group that X^{a2} represents is preferably a group not containing a hetero atom, more preferably an aliphatic hydrocarbon group not containing a hetero atom, or an alicyclic hydrocarbon group not containing a hetero atom, and even more preferably a group of a combination of an aliphatic hydrocarbon group not containing a hetero atom and an alicyclic hydrocarbon group not containing a hetero atom.

The structure represented by the general formula (a-3), the general formula (a-4) or the general formula (a-5) is, from the viewpoint of dielectric characteristics, preferably a structure represented by the following formula (a-3′) or formula (a-4′) or the following general formula (a-5′), respectively.

Among these, from the viewpoint of dielectric characteristics, more preferred is the structure represented by the following formula (a-4′) or the following general formula (a-5′), and even more preferred is the structure represented by the following general formula (a-5′).

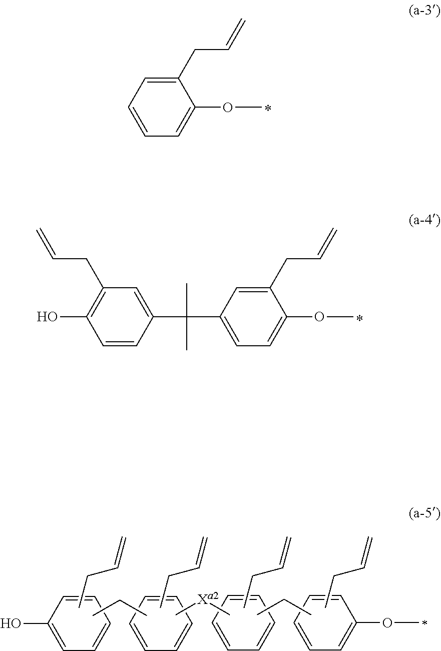

In the formulae, X^{a2} is the same as X^{a2} in the general formula (a-5). * indicates a bonding position to the other structure.

The component (A) is a polyphenylene ether derivative and therefore has a phenylene ether bond, and preferably has a structural unit represented by the following general formula (a-10).

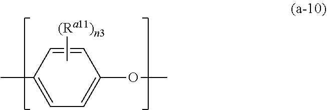

In the formula, R^{a11} is an aliphatic hydrocarbon group having 1 to 5 carbon atoms, or a halogen atom. n3 is an integer of 0 to 4.

Examples of the aliphatic hydrocarbon group having 1 to 5 carbon atoms that R an in the general formula (a-10) represents include a methyl group, an ethyl group, an n-propyl group, an isopropyl group, an n-butyl group, an isobutyl group, a t-butyl group and an n-pentyl group. The aliphatic hydrocarbon group is preferably an aliphatic hydrocarbon group having 1 to 3 carbon atoms, more preferably an alkyl group having 1 to 3 carbon atoms, even more preferably a methyl group.

Examples of the halogen atom that R an represents include a fluorine atom, a chlorine atom, a bromine atom and an iodine atom.

Among the above, R^{a11} is preferably an aliphatic hydrocarbon group having 1 to 5 carbon atoms.

n3 in the general formula (a-10) is an integer of 0 to 4, preferably an integer of 1 or 2, more preferably 2. In the case where n3 is 1 or 2, the substitution position of R^{a11} is preferably in an ortho-position based on the substitution position of the oxygen atom. When n3 is 2 or more, plural R^{a11}'s can be the same as or different from each other.

Specifically, the structural unit represented by the general formula (a-10) is preferably a structural unit represented by the following general formula (a-10′).

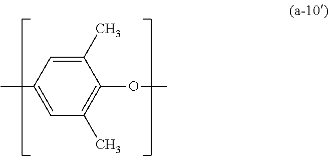

The component (A) preferably contains a polyphenylene ether derivative represented by any of the following general formulae (a-6) to (a-8), more preferably contains a polyphenylene ether derivative represented by the following general formula (a-7) or (a-8), even more preferably contains a polyphenylene ether derivative represented by the following general formula (a-8).

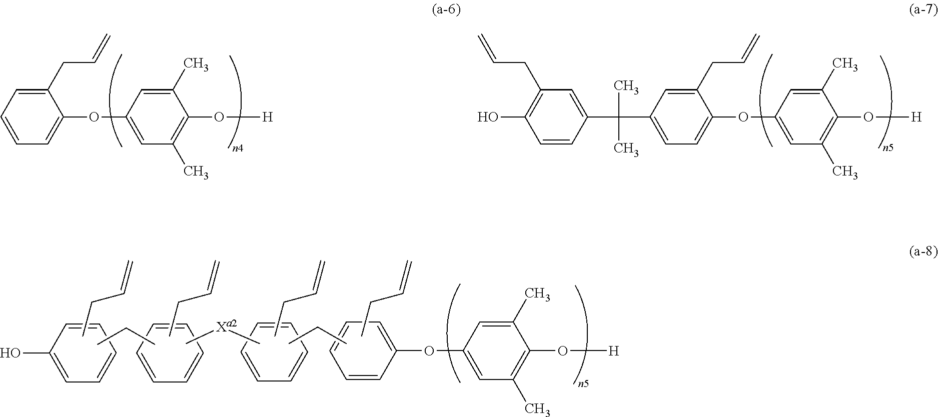

In the formulae, X^{a2} is the same as X^{a2} in the general formula (a-5). n4 to n6 each are independently an integer of 1 to 200.

In the general formulae (a-6) to (a-8), n4 to n6 each are independently an integer of 1 to 200, and are, from the viewpoint of dielectric characteristics and compatibility with other resins, preferably an integer of 1 to 150, more preferably an integer of 1 to 120, even more preferably an integer of 1 to 100.

The component (A) may be a mixture of polyphenylene ether derivatives differing in the value of n4 to n6 in the general formulae (a-6) to (a-8).

### [Number-Average Molecular Weight (Mn) of Component (A)]

Though not specifically limited, the number average molecular weight of the component (A) is preferably 1,000 to 25,000, more preferably 2,000 to 20,000, even more preferably 3,000 to 10,000, specially preferably 4,000 to 6,000.

When the number-average molecular weight of the component (A) is the above-mentioned lower limit or more, the dielectric characteristics tends to be better. When the number-average molecular weight of the component (A) is the above-mentioned upper limit or less, the compatibility of the resin composition becomes better, and even when left for a long period of time, the composition tends to hardly separate.

### [Production Method for Component (A)]

One embodiment of the production method for the component (A) is described below, which, however, is not limitative.

For example, the component (A) can be produced by redistribution reaction of a phenol compound containing a structure represented by any of the above-mentioned general formulae (a-1) to (a-5) and a polyphenylene ether in an organic solvent.

In the following description, a phenol compound containing a structure represented by any of the general formulae (a-1) to (a-5) may be referred to as “unsaturated aliphatic hydrocarbon group-containing phenol compound (1)”. The polyphenylene ether used as a raw material for the redistribution reaction may be referred to as “raw material polyphenylene ether”. The number-average molecular weight of the raw material polyphenylene ether is, though not specifically limited thereto, preferably 3,000 to 30,000.

The redistribution reaction is a reaction where the oxyradical in the unsaturated aliphatic hydrocarbon group-containing phenol compound (1) attacks the carbon atom to which the oxygen atom in the raw material polyphenylene ether bonds, whereby the O—C bond is cleaved at that position. At that time, the oxyradical in the unsaturated aliphatic hydrocarbon group-containing phenol compound (1) that have attacked bonds to the bond-cleaved carbon atom and is thereby taken in the structure of the polyphenylene ether. For the redistribution reaction, any known method can be used and applied.

The molecular weight of the component (A) can be controlled by the amount to be used of the unsaturated aliphatic hydrocarbon group-containing phenol compound (1), and when the amount used of the unsaturated aliphatic hydrocarbon group-containing phenol compound (1) is larger, the molecular weight of the component (A) is made smaller. In other words, the amount to be used of the unsaturated aliphatic hydrocarbon group-containing phenol compound (1) is appropriately adjusted in order that the number-average molecular weight of the component (A) to be finally produced can fall within a preferred range.

The amount to be used of the unsaturated aliphatic hydrocarbon group-containing phenol compound (1) is, though not specifically limited but for example, able to be determined depending on the number-average molecular weight of the raw material polyphenylene ether to be reacted with the unsaturated aliphatic hydrocarbon group-containing phenol compound (1).

For example, when the number average molecular weight of the raw material polyphenylene ether is 3,000 to 30,000, the amount of the hydroxy group of the unsaturated aliphatic hydrocarbon group-containing phenol compound (1) relative to one mol of the raw material polyphenylene ether is preferably 1 to 10 mols, more preferably 1 to 8 mols, even more preferably 2 to 6 mols. When the amount used of the unsaturated aliphatic hydrocarbon group-containing phenol compound (1) falls within the range, the component (A) whose number-average molecular weight falls within the above-mentioned preferred range can be produced.

Examples of the organic solvent used in the production process for the component (A) include an alcohol solvent such as methanol, ethanol, butanol, butyl cellosolve, ethylene glycol monomethyl ether, and propylene glycol monomethyl ether; a ketone solvent such as acetone, methyl ethyl ketone, methyl isobutyl ketone and cyclohexanone; an aromatic hydrocarbon solvent such as toluene, xylene and mesitylene; an ester solvent such as methoxyethyl acetate, ethoxyethyl acetate, butoxyethyl acetate and ethyl acetate; and a nitrogen atom-containing solvent such as N,N-dimethylformamide, N,N-dimethylacetamide and N-methyl-2-pyrrolidone.

One kind alone of the organic solvent or two or more kinds thereof can be used either singly or as combined.

In the production process for the component (A), as mentioned above, a reaction catalyst can be used as needed.

As the reaction catalyst, for example, from the viewpoint of obtaining the component (A) having a stable number-average molecular weight with good productivity, preferred is a combination use of an organic peroxide such as t-butylperoxyisopropyl monocarbonate and a metal carboxylate such as manganese naphthenate or manganese octylate.

In the case of a combination use of an organic peroxide and a metal carboxylate, the amount to be used of the two is, though not specifically limited thereto, preferably such that the amount to be used of the organic peroxide is 0.5 to 5 parts by mass and the amount to be used of the metal carboxylate is 0.05 to 0.5 parts by mass, relative to 100 parts by mass of the raw material polyphenylene ether. When the content of the organic peroxide and the metal carboxylate falls within the above range, the reaction speed in producing the component (A) tends to increase and gelation can be suppressed more effectively.

The unsaturated aliphatic hydrocarbon group-containing compound (1), the raw material polyphenylene ether, an organic solvent and optionally a reaction solvent are put into a reactor and reacted therein while optionally heated, thermally retained or stirred to produce the component (A).

The reaction temperature in the redistribution reaction is preferably 70 to 110° C. The reaction time for the redistribution reaction is preferably 1 to 8 hours. When the reaction temperature and the reaction time each fall within the above range, the workability may better, gelation can be suppressed, and the component (A) having the above-mentioned number-average molecular weight can be thereby readily produced. However, the reaction condition is not limited to the above-mentioned condition, and can be appropriately adjusted depending on the kind of the reaction materials. To the reaction condition, any known reaction condition for redistribution reaction can be applied.

The solid concentration during the reaction (hereinafter this may be referred to as “reaction concentration”) in the production process for the component (A) is, though not specifically limited, preferably 10 to 60% by mass, more preferably 15 to 55% by mass, even more preferably 20 to 50% by mass. When the reaction concentration is the above-mentioned lower limit or more, a good reaction speed can be attained and the productivity can be better. When the reaction concentration is the upper limit or less, good solubility can be attained, the stirring efficiency can improve, and gelation can be suppressed more effectively,

A solution of the polyphenylene ether derivative (A) produced according to the above-mentioned method can be optionally concentrated to remove a part of the reaction solvent therein, or can be diluted by adding an organic solvent thereto.

The resin composition of the present embodiment tends to have more excellent dielectric characteristics than a resin composition containing the above-mentioned, raw material polyphenylene ether in place of the component (A).

In the case where the resin composition of the present embodiment contains the component (A), the content thereof is, though not specifically limited but from the viewpoint of dielectric characteristics, preferably 0.1 to 20 parts by mass relative to 100 parts by mass of the sum total of the resin component in the resin composition, more preferably 0.5 to 10 parts by mass, even more preferably 0.7 to 5 parts by mass, especially preferably 1 to 3 parts by mass.

Here, in the present specification, “resin component” indicates, for example, the component (A), the component (B), the component (C) and the component (D), and in the case where the resin composition contains any other resin, the other resin is also contained in the resin component.

### <Maleimide Compound or Derivative Thereof (B)>

The component (B) is at least one selected from the group consisting of a maleimide compound having at least two N-substituted maleimide groups and a derivative thereof.

Examples of the “derivative of a maleimide compound having at least two N-substituted maleimide groups” include an addition reaction product between a maleimide compound having at least two N-substituted maleimide groups to be mentioned below, and a diamine compound (b2).

One kind alone of the component (B) or two or more kinds thereof can be used either singly or as combined.

The component (B) is, from the viewpoint of the compatibility with other resins, the adhesiveness to conductors and dielectric characteristics, preferably at least one compound selected from the following groups (i) and (ii).

(i) A maleimide compound (b1) having at least two N-substituted maleimide groups (hereinafter this may be referred to as “maleimide compound (b1)” or “component (b1)”).

(ii) An aminomaleimide compound having a structural unit derived from a maleimide compound (b1) and a structural unit derived from a diamine compound (b2) (hereinafter this may be referred to as “aminomaleimide compound (B1)” or “component (B1)”).

### (Maleimide Compound (b1) Having at Least Two N-Substituted Maleimide Groups)

The component (b1) is not specifically limited so far as it is a maleimide compound having at least two N-substituted maleimide groups.

One kind alone of the component (31) or two or more kinds thereof can be used either singly or as combined.

Examples of the component (b1) include an aromatic maleimide compound having two N-substituted maleimide groups in the molecule such as bis(4-maleimidophenyl)methane, bis(4-maleimidophenyl) ether, bis(4-maleimidophenyl) sulfone, 3,3′-dimethyl-5, 5′-diethyl-4,4′-diphenylmethanebismaleimide, 4-methyl-1,3-phenylenebismaleimide, m-phenylenebismaleimide, and 2,2-bis[4-(4-maleimidophenoxy)phenyl]propane; an aromatic polymaleimide compound having at least three N-substituted maleimide groups in the molecule such as polyphenylmethane maleimide, and biphenylaralkyl-type maleimide; and an aliphatic maleimide compound such as 1,6-bismaleimido-(2,2,4-trimethyl)hexane, and pyrophosphoric acid binder-type long-chain alkyl bismaleimide. Among these, from the viewpoint of the compatibility with other resins, the adhesiveness to conductors, heat resistance, low thermal expansion property and mechanical characteristics, preferred are an aromatic maleimide compound having two N-substituted maleimide groups in the molecule, and an aromatic polymaleimide compound having at least three N-substituted maleimide groups in the molecule; and more preferred are 3,3′-dimethyl-5,5′-diethyl-4,4′-diphenylmethanebismaleimide, and biphenylaralkyl-type maleimide.

As the component (b1), preferred is a compound represented by the following general formula (b1-1).

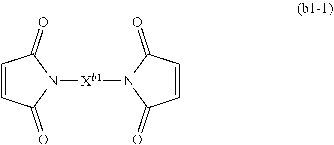

In the formula, X^{b1} is a divalent organic group.

X^{b1} in the general formula (b1-1) is a divalent organic group, and corresponds to a divalent group formed by removing two N-substituted maleimide groups from the component (b1).

Examples of the organic group that X^{b1} represents include a group represented by the following general formula (b1-2), a group represented by the following general formula (b1-3), a group represented by the following general formula (b1-4), a group represented by the following general formula (b1-5), and a group represented by the following general formula (b1-6).

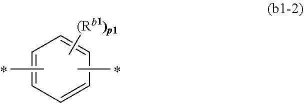

In the formula, R^{b1} is an aliphatic hydrocarbon group having 1 to 5 carbon atoms, or a halogen atom. p1 is an integer of 0 to 4. * indicates a bonding position to the other structure.

Examples of the aliphatic hydrocarbon group having 1 to 5 carbon atoms that R^{b1} represents include a methyl group, an ethyl group, an n-propyl group, an isopropyl group, an n-butyl group, an isobutyl group, a t-butyl group, and an n-pentyl group. The aliphatic hydrocarbon group is preferably an aliphatic hydrocarbon group having 1 to 3 carbon atoms, more preferably an alkyl group having 1 to 3 carbon atoms, even more preferably a methyl group.

The halogen atom includes a fluorine atom, a chlorine atom, a bromine atom, and an iodine atom.

p is an integer of 0 to 4, and is, from the viewpoint of easy availability, preferably an integer of 0 to 2, more preferably 0 or 1, even more preferably 0. In the case where p1 is an integer of 2 or more, plural R^{b1}'s can be the same as or different from each other.

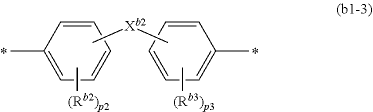

In the formula, R^{b2} and R^{b3} each independently are an aliphatic hydrocarbon group having 1 to 5 carbon atoms, or a halogen atom. X^{b2} is an alkylene group having 1 to 5 carbon atoms, an alkylidene group having 2 to 5 carbon atoms, an ether group, a sulfide group, a sulfonyl group, a carbonyloxy group, an keto group, a single bond, or a divalent group represented by the following general formula (b1-3-1). p2 and p3 each independently are an integer of 0 to 4. * indicates a bonding position to the other structure.

The aliphatic hydrocarbon group having 1 to 5 carbon atoms and the halogen atom that R^{b2} and R^{b3} represent can be the same as those of R^{b1}. The aliphatic hydrocarbon group is preferably an aliphatic hydrocarbon group having 1 to 3 carbon atoms, more preferably a methyl group or an ethyl group, even more preferably an ethyl group.

Examples of the alkylene group having 1 to 5 that X^{b2} represents include a methylene group, a 1,2-dimethylene group, a 1,3-trimethylene group, a 1,4-tetramethylene group, and a 1,5-pentamethylene group. The alkylene group is, from the viewpoint of the compatibility with other resins, the adhesiveness to conductors, heat resistance, low thermal expansion property and mechanical characteristics, preferred is an alkylene group having 1 to 3 carbon atoms, more preferred is an alkylene group having 1 or 2 carbon atoms, and even more preferred is a methylene group.

Examples of the alkylidene group having 2 to 5 carbon atoms that X^{b2} represents include an ethylidene group, a propylidene group, an isopropylidene group, a butylidene group, an isobutylidene group, a pentylidene group, and an isopentylidene group. Among these, from the viewpoint of the compatibility with other resins, the adhesiveness to conductors, heat resistance, low thermal expansion property and mechanical characteristics, preferred is an isopropylidene group.

p2 and p3 each independently are an integer of 0 to 4, and, from the viewpoint of easy availability, both are preferably an integer of 0 to 3, more preferably an integer of 0 to 2, even more preferably 0 or 2. In the case where p2 or p3 is an integer of 2 or more, plural R^{b2}'s or R^{b3}'s each can be the same as or different from each other.

The divalent group represented by the general formula (b1-3-1), which is represented by X^{b2}, is as mentioned below.

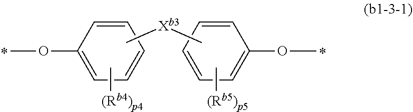

In the formula, R^{b4} and R^{b5} each independently are an aliphatic hydrocarbon group having 1 to 5 carbon atoms, or a halogen atom. X^{b3} is an alkylene group having 1 to 5 carbon atoms, an alkylidene group having 2 to 5 carbon atoms, an ether group, a sulfide group, a sulfonyl group, a carbonyloxy group, a keto group or a single bond. p4 and p5 each independently are an integer of 0 to 4. * indicates a bonding position to the other structure.

The aliphatic hydrocarbon group having 1 to 5 carbon atoms and the halogen atom that R^{b4} and R^{b5} represent can be described as the same as those for R^{b1}.

The alkylene group having 1 to 5 carbon atoms, and the alkylidene group having 2 to 5 carbon atoms, which X^{b3} represents, include the same as those of the alkylene group having 1 to 5 carbon atoms, and the alkylidene group having 2 to 5 carbon atoms, which X b2 represents.

Among the above-mentioned choices, X^{b3} is preferably an alkylidene group having 2 to 5 carbon atoms, more preferably an alkylidene group having 2 to 4 carbon atoms, even more preferably an isopropylidene group.

p4 and p5 each independently are an integer of 0 to 4, and from the viewpoint of easy availability, these are preferably an integer of 0 to 2, more preferably 0 or 1, even more preferably 0. In the case where p4 or p5 is an integer of 2 or more, plural R^{b4}'s or R^{b5}'s each can be the same as or different from each other.

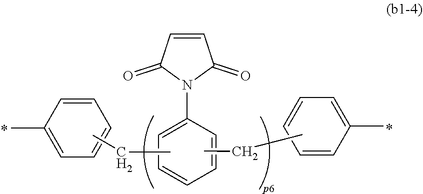

In the formula, p6 is an integer of 0 to 10. * indicates a bonding position to the other structure.

p6 is, from the viewpoint of easy availability, preferably an integer of 0 to 5, more preferably an integer of 0 to 4, even more preferably 0 to 3.

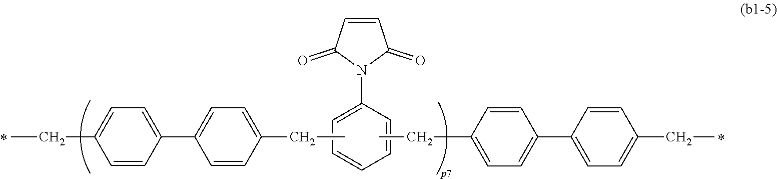

In the formula, p7 is a number of 0 to 5. * indicates a bonding position to the other structure.

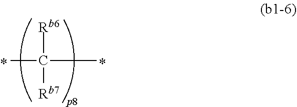

In the formula, R^{b6} and R^{b7} each independently are a hydrogen atom, or an aliphatic hydrocarbon group having 1 to 5 carbon atoms. p8 is an integer of 1 to 8. * indicates a bonding position to the other structure.

The aliphatic hydrocarbon group having 1 to 5 carbon atoms that R^{b6} and R^{b7} represent can be described as the same as that for R^{b1}.

p8 is an integer of 1 to 8, and is preferably an integer of 1 to 5, more preferably an integer of 1 to 3, even more preferably 1.

In the case where p8 is an integer of 2 or more, plural R^{b6}'s or R^{b7}'s each can be the same as or different from each other.

### (Aminomaleimide Compound (B1))

The component (B1) is an aminomaleimide compound having a structural unit derived from a maleimide compound (b1) and a structural unit derived from a diamine compound (b2).

One kind alone of the component (B1) or two or more kinds thereof can be used either singly or as combined.

[Structural Unit Derived from Maleimide Compound (b1)]

Examples of the structural unit derived from the component (b1) include a structural unit formed by Michael addition reaction between at least one N-substituted maleimide group of the N-substituted maleimide groups that the component (b1) has and the amino group that the diamine compound (b2) has.

The structural unit derived from the component (b1) contained in the component (B1) can be one kind alone of the structural unit or two or more kinds thereof.

Examples of the structural unit derived from the component (b1) include a group represented by the following general formula (b1-7) and a group represented by the following general formula (b1-8).

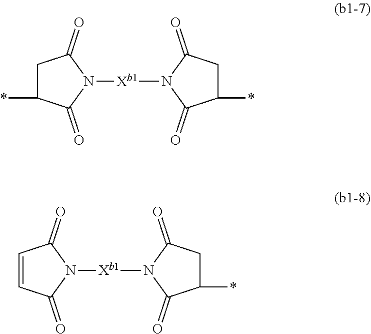

In the formulae, X^{b1} is a divalent organic group, and * indicates a bonding position to the other structure.

The description of X^{b1} in the general formula (b1-7) and the general formula (b1-8) is the same as that of X^{b1} in the above-mentioned general formula (b1-1).

Though not specifically limited, the content of the structural unit derived from the component (b1) in the aminomaleimide compound (B1) is preferably 5 to 95% by mass, more preferably 30 to 93% by mass, even more preferably 60 to 90% by mass, especially preferably 75 to 90% by mass. When the content of the structural unit derived from the component (b1) falls within the above range, dielectric characteristics and film handleability tend to be better.

[Structural Unit Derived from Diamine Compound (b2)]

Examples of the structural unit derived from the component (b2) include a structural unit formed by Michael addition reaction between one or both amino groups of the two amino groups that the component (b2) has and the N-substituted maleimide group that the maleimide compound (b1) has.

The structural unit derived from the component (b2) contained in the component (B1) can be one kind alone of the structural unit or two or more kinds thereof.

The amino group that the component (b2) has is preferably a primary amino group.

Examples of the structural unit derived from the component (b2) include a group represented by the following general formula (b2-1) and a group represented by the following general formula (b2-2).

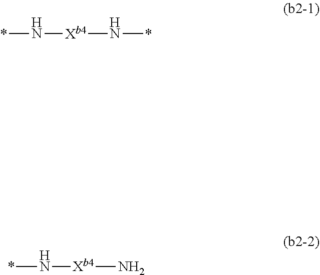

In the formulae, X^{b4} is a divalent organic group, and * indicates a bonding position to the other structure.

X^{b4} in the general formula (b2-1) and the general formula (b2-2) is a divalent organic group, and corresponds to the divalent group formed by removing two amino groups from the component (b2).

X^{b4} in the general formula (b2-1) and the general formula (b2-2) is preferably a divalent group represented by the following general formula (b2-3).

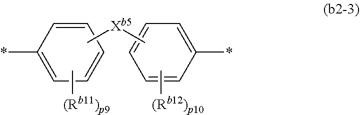

In the formula, R^{b11} and R^{b12} each independently are an aliphatic hydrocarbon group having 1 to 5 carbon atoms, an alkoxy group having 1 to 5 carbon atoms, a hydroxy group or a halogen atom. X^{b5} is an alkylene group having 1 to 5 carbon atoms, an alkylidene group having 2 to 5 carbon atoms, an ether group, a sulfide group, a sulfonyl group, a carbonyloxy group, a keto group, a fluorenylene group, a single bond, or a divalent group represented by the following general formula (b2-3-1) or (b2-3-2). p9 and p10 each independently are an integer of 0 to 4. * indicates a bonding position to the other structure.

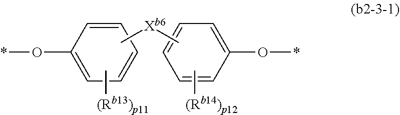

In the formula, R^{b13} and R^{b14} each independently are an aliphatic hydrocarbon group having 1 to 5 carbon atoms, or a halogen atom. X^{b6} is an alkylene group having 1 to 5 carbon atoms, an alkylidene group having 2 to 5 carbon atoms, an m-phenylenediisopropylidene group, a p-phenylenediisopropylidene group, an ether group, a sulfide group, a sulfonyl group, a carbonyloxy group, a keto group or a single bond. p11 and p12 each independently are an integer of 0 to 4. * indicates a bonding position to the other structure.

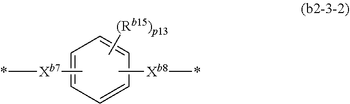

In the formula, R^{b15} is an aliphatic hydrocarbon group having 1 to 5 carbon atoms, or a halogen atom. X^{b7} and X^{b8} each independently are an alkylene group having 1 to 5 carbon atoms, an alkylidene group having 2 to 5 carbon atoms, an ether group, a sulfide group, a sulfonyl group, a carbonyloxy group, a keto group or a single bond. p13 is an integer of 0 to 4. * indicates a bonding position to the other structure.

The aliphatic hydrocarbon group having 1 to 5 carbon atoms and the halogen atom that R^{b11}, R^{b12}, R^{b13}, R^{b14} and R^{b15} in the general formula (b2-3), the general formula (b2-3-1) and the general formula (b2-3-2) represent include the same as those of R^{b1} in the general formula (b1-2). The aliphatic hydrocarbon group is preferably an aliphatic hydrocarbon group having 1 to 3 carbon atoms, more preferably an alkyl group having 1 to 3 carbon atoms, even more preferably a methyl group or an ethyl group.

The alkylene group having 1 to 5 carbon atoms and the alkylidene group having 2 to 5 carbon atoms that X^{b5} in the general formula (b2-3), X^{b6} in the general formula (b2-3-1) and X^{b7} and X^{b8} in the general formula (b2-3-2) represent can be described in the same manner as that for X^{b2} in the general formula (b1-3).

p9 and p10 in the general formula (b2-3) each independently are an integer of 0 to 4, and from the viewpoint of easy availability, these are preferably an integer of 0 to 3, more preferably an integer of 0 to 2, even more preferably 0 or 2.

In the case where p9 and p10 are an integer of 2 or more, plural R^{b11}'s or R^{b12}'s each can be the same as or different from each other.

p11 and p12 in the general formula (b2-3-1) each independently are an integer of 0 to 4, and from the viewpoint of easy availability, these are preferably an integer of 0 to 2, more preferably 0 or 1, even more preferably 0.

In the case where p11 and p12 are an integer of 2 or more, plural R^{b13}'s or R^{b14}'s each can be the same as or different from each other.

p13 in the general formula (b2-3-2) is an integer of 0 to 4, and is, from the viewpoint of easy availability, preferably an integer of 0 to 2, more preferably 0 or 1, even more preferably 0.

Examples of the component (b2) include 4,4′-diaminodiphenylmethane, 4,4′-diamino-3,3′-dimethyldiphenylmethane, 4,4′-diamino-3,3′-diethyldiphenylmethane, 4,4′-diaminodiphenyl ether, 4,4′-diaminodiphenyl sulfone, 3,3′-diaminodiphenyl sulfone, 4,4′-diaminodiphenyl ketone, 4,4′-diaminobiphenyl, 3,3′-dimethyl-4,4′-diaminobiphenyl, 2,2′-dimethyl-4,4′-diaminobiphenyl, 3,3′-dihydroxybenzidine, 2,2-bis(3-amino-4-hydroxyphenyl)propane, 3,3′-dimethyl-5, 5′-diethyl-4,4′-diaminodiphenylmethane, 2,2-bis(4-aminophenyl)propane, 2,2-bis [4-(4-aminophenoxy)phenyl]propane, 1,3-bis(3-aminophenoxy)benzene, 1,3-bis(4-aminophenoxy)benzene, 1,4-bis(4-aminophenoxy)benzene, 4,4′-bis(4-aminophenoxy)biphenyl, 1,3-bis[1-[4-(4-aminophenoxy)phenyl]-1-methylethyl]benzene, 1,4-bis[1-[4-(4-aminophenoxy)phenyl]-1-methylethyl]benzene, 4,4′-[1,3-phenylenebis(1-methylethylidene)]bisaniline, 4,4′-[1,4-phenylenebis(1-methylethylidene)]bisaniline, 3,3′-[1,3-phenylenebis(1-methylethylidene)]bisaniline, bis[4-(4-aminophenoxy)phenyl]sulfone, bis[4-(3-aminophenoxy)phenyl]sulfone, and 9,9-bis(4-aminophenyl)fluorene.

Among these, as the component (b2), from the viewpoint that they are excellent in solubility in organic solvent, reactivity with the maleimide compound (b1) and heat resistance, preferred are 4,4′-diaminodiphenylmethane, 4,4′-diamino-3,3′-dimethyldiphenylmethane, 4,4′-diamino-3,3′-diethyldiphenylmethane, 2,2-bis[4-(4-aminophenoxy)phenyl]propane, 4,4′-[1,3-phenylenebis(1-methylethylidene)]bisaniline, and 4,4′-[1,4-phenylenebis(1-methylethylidene)]bisaniline. Also as the component (b2), from the viewpoint that it is excellent in dielectric characteristics and low water absorption property, preferred is 3,3′-dimethyl-5,5′-diethyl-4,4′-diaminodiphenylmethane. Also as the component (b2), from the viewpoint that it is excellent in high adhesiveness to conductors, and in mechanical characteristics such as elongation and strength at break, preferred is 2,2-bis[4-(4-aminophenoxy)phenyl]propane. Further, as the component (b2), from the viewpoint that they are excellent in solubility in organic solvent, reactivity in synthesis, heat resistance, and high adhesiveness to conductors, and are additionally excellent in dielectric characteristics and low moisture absorption property, preferred are 4,4′-[1,3-phenylenebis(1-methylethylidene)]bisaniline, and 4,4′-[1,4-phenylenebis(1-methylethylidene)]bisaniline.

Though not specifically limited, the content of the structural unit derived from the component (b2) in the aminomaleimide compound (B1) is preferably 5 to 95% by mass, more preferably 7 to 70% by mass, even more preferably 10 to 40% by mass, especially preferably 10 to 25% by mass. When the content of the structural unit derived from the component (b2) falls within the range, dielectric characteristics, heat resistance, flame resistance and glass transition temperature tend to be better.

Though not specifically limited, in the aminomaleimide compound (B1), the equivalent ratio (Tb2/Tb1) of the total equivalent (Tb2) of the group derived from —NH₂ (including —NH₂) in the diamine compound (b2) to the total equivalent (Tb1) of the group derived from the N-substituted maleimide group (including N-substituted maleimide group) in the maleimide compound (b1) is preferably 0.05 to 10, more preferably 0.5 to 7, even more preferably 1 to 5. When the equivalent ratio (Tb2/Tb1) falls within the range, dielectric characteristics, heat resistance, flame resistance and glass transition temperature tend to be better.

The number-average molecular weight of the aminomaleimide compound (B1) is, not specifically limited thereto, preferably 400 to 10,000, more preferably 500 to 5,000, even more preferably 600 to 2,000, especially preferably 700 to 1,500.

From the viewpoint of dielectric characteristics, solubility in organic solvent, high adhesiveness to conductors, and moldability into resin films, the aminomaleimide compound (B1) preferably contains an aminomaleimide compound represented by the following general formula (b2-4).

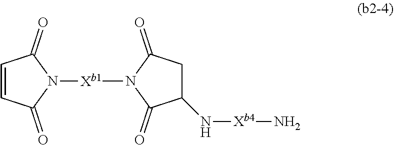

In the formula, X″ and X″ are as described above.

### (Production Method for Aminomaleimide Compound (B1))

The component (B1) can be produced, for example, by reacting a maleimide compound (b1) and a diamine compound (b2) in an organic solvent.

By reacting a maleimide compound (b1) and a diamine compound (b2), there can be obtained the aminomaleimide compound (B1) through Michael addition reaction of the maleimide compound (b1) and the diamine compound (b2).

In reacting the maleimide compound (b1) and the diamine compound (b2), a reaction catalyst can be used as needed.

Examples of the reaction catalyst include an acid catalyst such as p-toluenesulfonic acid; an amine such as triethylamine, pyridine and tributylamine; an imidazole such as methyl imidazole and phenylimidazole; and a phosphorus-based catalyst such as triphenyl phosphine.

One kind alone of reaction catalyst or two or more kinds thereof can be used either singly or as combined.

Not specifically limited, the blending amount of the reaction catalyst can be, for example, 0.01 to 5 parts by mass relative to 100 parts by mass of the total amount of the maleimide compound (b1) and the diamine compound (b2).

The reaction temperature in the above-mentioned reaction is, from the viewpoint of workability such as reaction speed, and from the viewpoint of suppressing gelation during reaction, preferably 50 to 160° C. Also the reaction time for the reaction is, from the same viewpoint, preferably 1 to 10 hours.

In this step, the solid concentration of the reaction material and the solution viscosity can be adjusted by adding a reaction solvent or by concentration. The solid concentration of the reaction material is, though not specifically limited, preferably 10 to 90% by mass, more preferably 15 to 85% by mass, even more preferably 20 to 80% by mass. When the solid concentration of the reaction material is not lower than the lower limit, a good reaction speed can be attained to better productivity. When the solid concentration of the reaction material is not higher than the upper limit, good solubility can be attained to improve stirring efficiency, and gelation can be suppressed more favorably.

In the case where the resin composition of the present embodiment contains the component (B), the content thereof is, though not specifically limited but from the viewpoint of heat resistance and dielectric characteristics, preferably 10 to 90 parts by mass relative to 100 parts by mass of the sum total of the resin component in the resin composition, more preferably 20 to 80 parts by mass, even more preferably 30 to 70 parts by mass, especially preferably 40 to 60 parts by mass.

### <Conjugated Diene Polymer or Modified Conjugated Diene Polymer (C)>

The component (C) is, though not specifically limited thereto but preferably:

(c1) a conjugated diene polymer having a vinyl group in the side chain [hereinafter this may be referred to as “component (c1)”], or

(C1) a modified conjugated diene polymer produced by modifying (c1) a conjugated diene polymer having a vinyl group in the side chain with (c2) a maleimide compound having at least two N-substituted maleimide groups [hereinafter this may be referred to as “component (c2)”] [hereinafter the modified polymer may be referred to as “modified conjugated diene polymer (C1)” or “component (C1)”].

One kind alone of the component (C) or two or more kinds thereof can be used either singly or as combined.

((c1) Conjugated Diene Polymer Having Vinyl Group in Side Chain)

The component (c1) is not specifically limited so far as it is a conjugated diene polymer having a vinyl group in the side chain.

One kind alone of the component (c1) or two or more kinds thereof can be used either singly or as combined.

The component (c1) is preferably a conjugated diene polymer having plural vinyl groups in the side chain. The number of the vinyl groups that the component (c1) has in one molecule is, though not specifically limited but from the viewpoint of dielectric characteristics and heat resistance, preferably 3 or more, more preferably 5 or more, even more preferably 10 or more.

In the present specification, the conjugated diene polymer means a polymer of a conjugated diene compound.

Examples of the conjugated diene compound include 1,3-butadiene, isoprene, 1,3-pentadiene, 2,3-dimethyl-1,3-butadiene, 2-phenyl-1,3-butadiene, and 1,3-hexadiene.

The conjugated diene polymer can be a polymer of one kind of a conjugated diene compound, or can be a copolymer of two or more kinds of conjugated diene compounds.

In addition, the conjugated diene polymer can also be replaced with a copolymer of one or more kinds of conjugated diene compounds and one or more kinds of other monomers than conjugated diene compounds. The polymerization mode in that case is not specifically limited, and can be any of random polymerization, block polymerization or graft polymerization.

Specific examples of the component (c1) include a polybutadiene having a 1,2-vinyl group, a butadiene-styrene copolymer having a 1,2-vinyl group, and a polyisoprene having a 1,2-vinyl group. Among these, from the viewpoint of dielectric characteristics and heat resistance, preferred are a polybutadiene having a 1,2-vinyl group and a butadiene-styrene copolymer having a 1,2-vinyl group, and more preferred is a polybutadiene having a 1,2-vinyl group. As the polybutadiene having a 1,2-vinyl group, preferred is a polybutadiene homopolymer having a 1,2-vinyl group.

The butadiene-derived 1,2-vinyl group that the component (c1) has is a vinyl group contained in a butadiene-derived structural unit represented by the following formula (c1-1).

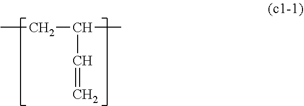

In the case where the component (c1) is a polybutadiene having a 1,2-vinyl group, the content of the structural unit having a 1,2-vinyl group relative to all the structural units derived from butadiene that constitutes the polybutadiene [hereinafter this may be referred to as “vinyl group content”] is, though not specifically limited but from the viewpoint of the compatibility with other resins, dielectric characteristics, low thermal expansion property and heat resistance, preferably 50 mol % or more, more preferably 60 mol % or more, even more preferably 70 mol % or more, especially preferably 80 mol % or more, and is most preferably 85 mol % or more. The upper limit of the vinyl group is not specifically limited, and can be 100 mol % or less, or can be 95 mol % or less, or can be 90 mol % or less. The structural unit having a 1,2-vinyl group is preferably a butadiene-derived structural unit represented by the above-mentioned formula (c1-1).

From the same viewpoint, the polybutadiene having a 1,2-vinyl group is preferably a 1,2-polybutadiene homopolymer.

Not specifically limited, the number-average molecular weight of the component (c1) is, from the viewpoint of the compatibility with other resins, dielectric characteristics, low thermal expansion property and heat resistance, preferably 400 to 2,500, more preferably 500 to 2,000, even more preferably 600 to 1,800, especially preferably 700 to 1,500.

### (Modified Conjugated Diene Polymer (C1))

The component (C1) is a modified conjugated diene polymer produced by modifying (c1) a conjugated diene polymer having a vinyl group in the side chain with (c2) a maleimide compound having at least two N-substituted maleimide groups.

[(c2) Maleimide Compound Having at Least Two N-Substituted Maleimide Groups]

The component (c2) can be any and every maleimide compound having at least two N-substituted maleimide groups, and for this, usable are those mentioned hereinabove for the maleimide compound or a derivative (B) thereof.

One kind alone of the component (c2) or two or more kinds thereof can be used either singly or as combined.

Among these, as the component (c2), from the viewpoint of solubility in organic solvent, gelation suppression during reaction, compatibility of the component (C1) with other resins, dielectric characteristics, low thermal expansion property and heat resistance, preferred is an aromatic bismaleimide compound substituted with an aliphatic hydrocarbon group, and more preferred is a compound represented by the following general formula (c2-1).

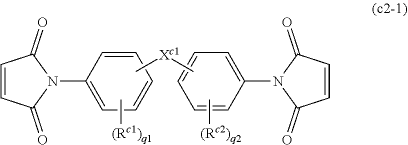

In the formula, R^{c1} and R^{c2} each independently are an aliphatic hydrocarbon group having 1 to 5 carbon atoms. X^{c1} is an alkylene group having 1 to 5 carbon atoms, an alkylidene group having 2 to 5 carbon atoms, an ether group, a sulfide group, a sulfonyl group, a carbonyloxy group, a keto group, a single bond, or a divalent group represented by the following general formula (c2-1-1). q1 and q2 each independently are an integer of 0 to 4, and q1+q2 is an integer of 1 or more.

Examples of the aliphatic hydrocarbon group having 1 to 5 carbon atoms that R^{c1} and R^{c2} represent include a methyl group, an ethyl group, an n-propyl group, an isopropyl group, an n-butyl group, an isobutyl group, a t-butyl group and an n-pentyl group. The aliphatic hydrocarbon group is, from the viewpoint of compatibility with other resins and gelation suppression during reaction, preferably an aliphatic hydrocarbon group having 1 to 3 carbon atoms, more preferably an alkyl group having 1 to 3 carbon atoms, even more preferably a methyl group or an ethyl group.

Examples of the alkylene group having 1 to 5 carbon atoms that X^{c1} represents include a methylene group, a 1,2-dimethylene group, a 1,3-trimethylene group, a 1,4-tetramethylene group, and a 1,5-pentamethylene group. The alkylene group is preferably an alkylene group having 1 to 3 carbon atoms, more preferably an alkylene group having 1 or 2 carbon atoms, even more preferably a methylene group.

Examples of the alkylidene group having 2 to 5 carbon atoms that X^{c1} represents include an ethylidene group, a propylidene group, an isopropylidene group, a butylidene group, an isobutylidene group, a pentylidene group, and an isopentylidene group.

q1 and q2 each independently are an integer of 0 to 4, and from the viewpoint of easy availability, compatibility with other resins and gelation suppression during reaction, both are preferably an integer of 1 to 3, more preferably 1 or 2, even more preferably 2.

From the same viewpoint, q1+q2 is preferably an integer of 1 to 8, more preferably an integer of 2 to 6, even more preferably 4.

In the case where q1 or q2 is an integer of 2 or more, plural R^{c1}'s or R^{c2}'s each can be the same as or different from each other.

The divalent group represented by the general formula (c2-1-1) that X^{c1} represents is as follows.

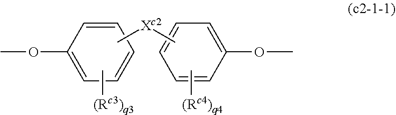

In the formula, R^{c3} and R^{c4} each independently are an aliphatic hydrocarbon group having 1 to 5 carbon atoms, or a halogen atom. X^{c2} represents an alkylene group having 1 to 5 carbon atoms, an alkylidene group having 2 to 5 carbon atoms, an ether group, a sulfide group, a sulfonyl group, a carbonyloxy group, a keto group or a single bond. q3 and q4 each independently are an integer of 0 to 4.

The aliphatic hydrocarbon group having 1 to 5 carbon atoms and the halogen atom that R^{c3} and R^{c4} represent can be described in the same manner as that for R^{c1}.

The alkylene group having 1 to 5 carbon atoms and the alkylidene group having 2 to 5 carbon atoms that X^{c2} represents include the same ones as those of the alkylene group having 1 to 5 carbon atoms and the alkylidene group having 2 to 5 carbon atoms that X^{c1} represents.

q3 and q4 each independently are an integer of 0 to 4, and from the viewpoint of easy availability, both are preferably an integer of 0 to 2, more preferably 0 or 1, even more preferably 0. In the case where q3 or q4 is an integer of 2 or more, plural R^{c3}'s or R^{c4}'s each can be the same as or different from each other.

As the compound represented by the general formula (c2-1), from the viewpoint of solubility in organic solvent, gelation suppression during reaction, and also from the viewpoint of the compatibility of the resultant component (C1) with other resins, dielectric characteristics, low thermal expansion property and heat resistance, preferred is 3,3′-dimethyl-5,5′-diethyl-4,4′-diphenylmethanebismaleimide.

The modified conjugated diene polymer (C1) preferably has a substituent formed by reaction of the vinyl group that the conjugated diene polymer (c1) has and the N-maleimide group that the maleimide compound (c2) has [hereinafter the substituent may be referred to as “substituent (x)”].

From the viewpoint of compatibility with other resins, dielectric characteristics, low thermal expansion property and heat resistance, the substituent (x) is preferably a group containing a structure represented by the following general formula (C-11) or (C-12) as the structure derived from the maleimide compound (c2).

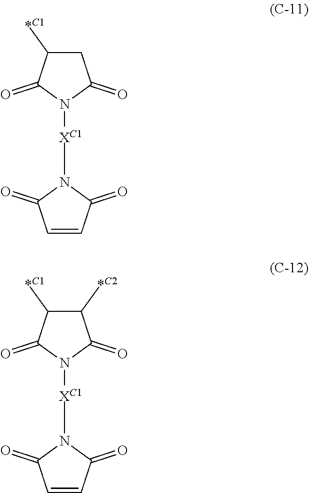

In the formulae, X^{C1} is a divalent organic group, *^{C1} is a site at which the group bonds to the carbon atom derived from the vinyl group that the conjugated diene polymer (c1) has in the side chain. *^{C2} is a site at which the group bonds to the other atom.

The description of X^{C1} in the general formulae (C-11) and (C-12) is the same as the description of X^{b1} in the above-mentioned general formula (b1-1).

More preferably, from the viewpoint of compatibility with other resins, dielectric characteristics, low thermal expansion property and heat resistance, the substituent (x) contains at least one selected from the group consisting of a structure represented by the following general formula (C-21) and a structure represented by the following general formula (c-22), as the structure derived from the maleimide compound (c2).

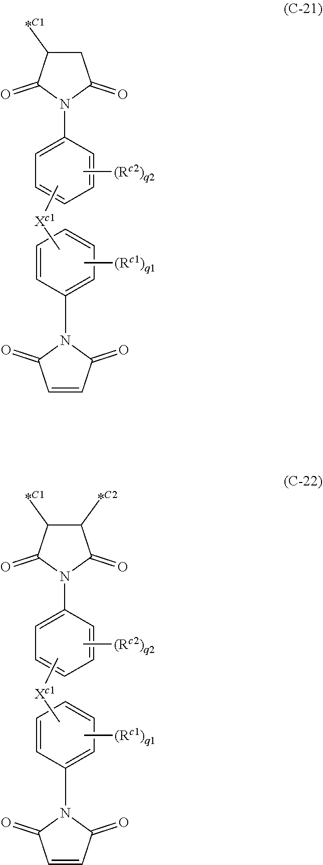

In the formulae, the description of R^{c1}, R^{c2}, X^{c1}, q1 and q2 is the same as in the above-mentioned general formula (c2-1). The description of *^{C1} and *^{C2} is the same as in the general formulae (C-11) and (C-12).

Preferably, the modified conjugated diene polymer (C1) has a substituent (x) and a vinyl group (y) in the side chain.

How many the substituents (x) are present in the modified conjugated diene polymer (C1) can be an index of how much the vinyl group of the component (c1) is modified with the component (c2) [hereinafter this may be referred to as “vinyl group modification ratio”].

Though not specifically limited, the vinyl group modification ratio is, from the viewpoint of compatibility with other resins, dielectric characteristics, low thermal expansion property and heat resistance, preferably 20 to 70%, more preferably 30 to 60%, even more preferably 35 to 50%. Here, the vinyl group modification ratio is a value determined according to the method described in Examples.

The vinyl group (y) is preferably a 1,2-vinyl group that the structural unit derived from butadiene has.

### (Production Method for Modified Conjugated Diene Polymer (C1))

The component (C1) can be produced by reacting a conjugated diene polymer (c1) and a maleimide compound (c2).

The method for reacting a conjugated diene polymer (c1) and a maleimide compound (c2) is not specifically limited. For example, a conjugated diene polymer (c1), a maleimide compound (c2), a reaction catalyst and an organic solvent are put into a reactor and reacted therein optionally with heating, thermal retaining and stirring to give the component (C1).

The reaction temperature for the reaction is, from the viewpoint of workability and gelation suppression during reaction, preferably 70 to 120° C., more preferably 80 to 110° C., even more preferably 85 to 105° C.

The reaction time for the reaction is, from the same viewpoint, preferably 0.5 to 15 hours, more preferably 1 to 10 hours, even more preferably 3 to 7 hours.

However, these reaction conditions can be appropriately adjusted depending on the kind of the raw materials used, and are not specifically limited.

Examples of the organic solvent to be used in the reaction include an alcohol solvent such as methanol, ethanol, butanol, butyl cellosolve, ethylene glycol monomethyl ether, and propylene glycol monomethyl ether; a ketone solvent such as acetone, methyl ethyl ketone, methyl isobutyl ketone, and cyclohexanone; an aromatic hydrocarbon solvent such as toluene, xylene and mesitylene; an ester solvent such as methoxyethyl acetate, ethoxyethyl acetate, butoxyethyl acetate, and ethyl acetate; and a nitrogen atom-containing solvent such as N,N-dimethylformamide, N,N-dimethylacetamide, and N-methyl-2-pyrrolidone.

One kind alone of organic solvent or two or more kinds thereof can be used either singly or as combined. Among the above, toluene is preferred from the viewpoint of resin solubility.

In the case where the above reaction is carried out in an organic solvent, the total content of the conjugated diene polymer (c1) and the maleimide compound (c2) in the reaction solution is, though not specifically limited, preferably 10 to 70% by mass, more preferably 15 to 60% by mass, even more preferably 20 to 50% by mass. When the total content of the conjugated diene polymer (c1) and the maleimide compound (c2) is not lower than the lower limit, good reaction speed tends to be attained to better productivity. When the total content of the conjugated diene polymer (c1) and the maleimide compound (c2) is not more than the upper limit, better solubility tends to be attained to improve stirring efficiency, and gelation tends to be suppressed more effectively.

From the viewpoint of suppressing gelation during reaction to attain sufficient reactivity, the reaction catalyst is preferably an organic peroxide, and more preferably α,α′bis(t-butylperoxy)diisopropylbenzene.

One kind alone of reaction catalyst or two or more kinds thereof can be used either singly or as combined.

Though not specifically limited, the amount to be used of the reaction catalyst is preferably 0.01 to 1.2 parts by mass relative to 100 parts by mass of the sum total of the conjugated diene polymer (c1) and the maleimide compound (c2), more preferably 0.03 to 1.0 part by mass, even more preferably 0.05 to 0.8 parts by mass.

In carrying out the reaction, the ratio (Mₘ/Mᵥ) of the molar number (Mₘ) of the N-substituted maleimide group that the maleimide compound (c2) has, to the molar number (Mᵥ) of the side chain vinyl group that the conjugated diene polymer (c1) has is, though not specifically limited but from the viewpoint of the compatibility of the resultant component (C1) to other resins and gelation suppression during the reaction, preferably 0.001 to 0.5, more preferably 0.005 to 0.1, even more preferably 0.008 to 0.05.

The number-average molecular weight of the component (C) is, though not specifically limited but from the viewpoint of compatibility with other resins, dielectric characteristics, low thermal expansion property and heat resistance, preferably 700 to 6,000, more preferably 800 to 5,000, even more preferably 900 to 4,500, especially preferably 1,000 to 4,000.

In the case where the resin composition of the present embodiment contains the component (C), the content thereof is, though not specifically limited but from the viewpoint of compatibility with other resins, dielectric characteristics, low thermal expansion property and heat resistance, preferably 1 to 50 parts by mass relative to 100 parts by mass of the sum total of the resin component in the resin composition, more preferably 5 to 40 parts by mass, even more preferably 10 to 30 parts by mass, especially preferably 15 to 25 parts by mass.

### <Styrene-Based Thermoplastic Elastomer (D)>

The component (D) is not specifically limited so far as it is a thermoplastic elastomer having a structural unit derived from a styrene compound.

One kind alone of the component (D) or two or more kinds thereof can be used either singly or as combined.

As the component (D), preferred is one having a structural unit represented by the following general formula (d-1).

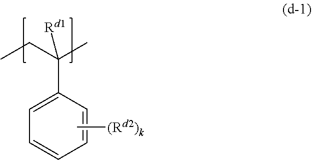

In the formula, R^{d1} is a hydrogen atom, or an alkyl group having 1 to 5 carbon atoms, and R^{d2} is an alkyl group having 1 to 5 carbon atoms. k is an integer of 0 to 5.

Examples of the alkyl group having 1 to 5 carbon atoms that R^{d1} and R^{d2} represent include a methyl group, an ethyl group and an n-propyl group. Among these, preferred is an alkyl group having 1 to 3 carbon atoms, more preferred is an alkyl group having 1 or 2 carbon atoms, and even more preferred is a methyl group.

k is preferably an integer of 0 to 2, more preferably 0 or 1, even more preferably 0.

Examples of the structural unit other than the structural unit derived from a styrene compound that the component (D) has include a butadiene-derived structural unit, an isoprene-derived structural unit, a maleic acid-derived structural unit and a maleic anhydride-derived structural unit.

The butadiene-derived structural unit and the isoprene-derived structural unit can be hydrogenated. In the case where the unit is hydrogenated, the butadiene-derived structural unit becomes a mixed structural unit of an ethylene unit and a butylene unit, and the isoprene-derived structural unit becomes a mixed structural unit of an ethylene unit and a propylene unit.

As the component (D), from the viewpoint of dielectric characteristics, adhesiveness to conductors, heat resistance, glass transition temperature and low thermal expansion property, preferred is at least one selected from the group consisting of a styrene-butadiene-styrene block copolymer hydrogenate (SEBS, SBBS), a styrene-isoprene-styrene block copolymer hydrogenate (SEPS) and a styrene-maleic anhydride copolymer (SMA), more preferred is at least one selected from the group consisting of a styrene-butadiene-styrene block copolymer hydrogenate (SEBS), and a styrene-isoprene-styrene block copolymer hydrogenate (SEPS), and even more preferred is a styrene-butadiene-styrene block copolymer hydrogenate (SEBS).

In the SEBS, the content of the styrene-derived structural unit [hereinafter this may be referred to as “styrene content”] is, though not specifically limited but from the viewpoint of dielectric characteristics, adhesiveness to conductors, heat resistance, glass transition temperature and low thermal expansion property, preferably 5 to 80% by mass, more preferably 10 to 75% by mass, even more preferably 15 to 70% by mass, especially preferably 20 to 50% by mass.

Though not specifically limited, the melt flow rate (MFR) of SEBS is, under the condition of 230° C. and a load of 2.16 kgf (21.2 N), preferably 0.1 to 20 g/10 min, more preferably 0.3 to 17 g/10 min, even more preferably 0.5 to 15 g/10 min.

Examples of commercial products of SEBS include Tuftec (registered trademark) H series and M series by Asahi Kasei Corporation, Septon (registered trademark) series by Kuraray Co., Ltd., and Krayton (registered trademark) G polymer series by Krayton Corporation.

Though not specifically limited, the weight-average molecular weight (Mw) of the component (D) is preferably 12,000 to 1,000,000, more preferably 30,000 to 500,000, even more preferably 50,000 to 120,000, especially preferably 70,000 to 100,000. The weight-average molecular weight (Mw) means a value measured in terms of polystyrene by gel permeation chromatography (GPC).

In the case where the resin composition (D) of the present embodiment contains the component (D), the content thereof is, though not specifically limited but from the viewpoint of dielectric characteristics, heat resistance, moldability and compatibility, preferably 10 to 60 parts by mass relative to 100 parts by mass of the sum total of the resin component in the resin composition, more preferably 15 to 50 parts by mass, even more preferably 20 to 40 parts by mass, especially preferably 25 to 30 parts by mass.

### <Imidazole Compound or Modified Product Thereof (E)>

The resin composition of the present embodiment contains the component (E), by which the composition tends to express further more excellent dielectric characteristics while having good heat resistance.

One kind alone of the component (E) or two or more kinds thereof can be used either singly or as combined.

Examples of the component (E) include imidazole compounds such as 2-methylimidazole, 2-ethylimidazole, 2-undecylimidazole, 2-heptadecylimidazole, 2-phenylimidazole, 1,2-dimethylimidazole, 2-ethyl-1-methylimidazole, 1,2-diethylimidazole, 1-ethyl-2-methylimidazole, 2-ethyl-4-methylimidazole, 4-ethyl-2-methylimidazole, 1 isobutyl-2-methylimidazole, 2-phenyl-4-methylimidazole, 1-benzyl-2-phenylimidazole, 1-cyanoethyl-2-methylimidazole, 1-cyanoethyl-2-ethylimidazole, 1-cyanoethyl-2-phenylimidazole, 1-cyanoethyl-2-ethyl-4-methylimidazole, 2-phenyl-4, 5-dihydroxymethylimidazole, 2-phenyl-4-methyl-5-hydroxymethylimidazole, 2,3-dihydro-1H-pyrrolo[1,2-a]benzimidazole, 2,4-diamino-6-[2 ‘-methylimidazolyl-(1’)]ethyl-s-triazine, 2,4-diamino-6-[2′-undecylimidazolyl-(1′)]ethyl-s-triazine, and 2,4-diamino-6-[2 ‘-ethyl-4’-methylimidazolyl-(1′)]ethyl-s-triazile; and modified imidazole compounds such as isocyanate-masked imidazole, epoxy-masked imidazole, salts of the above-mentioned imidazole compound with trimellitic acid, salts of the imidazole compound with isocyanuric acid, and salts of the imidazole compound with hydrobromic acid.

The modified imidazole compound is preferably a modified product of the imidazole compound represented by the following general formula (e-1) or the following general formula (e-2).

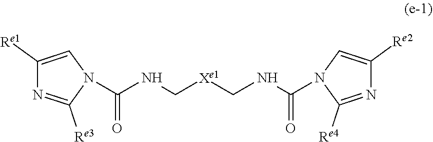

In the formula, R^{e1}, R^{e2}, R^{e3} and R^{e4} each independently are a hydrogen atom, an aliphatic hydrocarbon group having 1 to 20 carbon atoms, or a phenyl group, and X^{e1} is an alkylene group or a divalent aromatic hydrocarbon group.

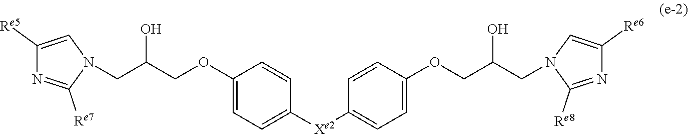

In the formula, R^{e5}, R^{e6}, R^{e7} and R^{e8} each independently are a hydrogen atom, an aliphatic hydrocarbon group having 1 to 20 carbon atoms, or a phenyl group, and X^{e2} is an alkylene group, an alkylidene group, an ether group or a sulfonyl group.

The resin composition of the present embodiment contains the modified imidazole compound represented by the general formula (e-1) or the general formula (e-2), and therefore the resin component therein can be further more excellent in compatibility. Though not clear, the reason can be presumed as follows. The modified imidazole compound has an imidazolyl group as a group having high polarity, and a hydrocarbon group represented by —CH₂—X^{e1}—CH₂— in the general formula (e-1) and -Ph-X^{e2}-Ph- in the general formula (e-2) as a group having low polarity. Consequently, the modified imidazole compound is presumed to function as a compatibilizer between the high-polarity component (B) and the low-polarity elastomer.

The resin composition containing the modified imidazole compound represented by the general formula (e-1) tends to have especially excellent dielectric characteristics while having good heat resistance. The resin composition containing the modified imidazole compound represented by the general formula (e-2) tends to be especially excellent in heat resistance while having improved dielectric characteristics.

The carbon number of the aliphatic hydrocarbon group that R^{e1}, R^{e2}, R^{e3} and R^{e4} represent in the general formula (e-1) is 1 to 20, preferably 1 to 10, more preferably 1 to 5, even more preferably 1 or 2.

Examples of the aliphatic hydrocarbon group that R^{e1}, R^{e2}, R^{e3} and R^{e4} represent include an alkyl group such as a methyl group, an ethyl group, a propyl group, a butyl group, a pentyl group, a hexyl group, a heptyl group, an octyl group, a decyl group, a dodecyl group, a tridecyl group, a tetradecyl group, a pentadecyl group, a hexadecyl group, a heptadecyl group and a stearyl group; an alkenyl group; and an alkynyl group. These aliphatic hydrocarbon groups can be linear or branched. Among these, preferred are a methyl group and an ethyl group.

The carbon number of the alkylene group that X^{e1} in the general formula (e-1) represents is preferably 1 to 10, more preferably 2 to 8, even more preferably 3 to 5.

Examples of the alkylene group that X^{e1} represents include a methylene group, an ethylene group, a 1,3-trimethylene group, a 1,4-tetramethylene group, a 1,5-pentamethylene group, and a 1,6-hexamethylene group. Among these, preferred is a 1,4-tetramethylene group.

The carbon number of the divalent aromatic hydrocarbon group that X^{e1} represents is preferably 6 to 20, more preferably 6 to 15, even more preferably 6 to 12.

Examples of the divalent aromatic group that X^{e1} represents include a phenylene group, a biphenylene group, a terphenylene group, a naphthylene group, and an anthrylene group.

The carbon number of the aliphatic hydrocarbon group that R^{e5}, R^{e6}, R^{e7} and R^{e8} represent in the general formula (e-2) is 1 to 20, preferably 1 to 10, more preferably 1 to 5, even more preferably 1 or 2.

Examples of the aliphatic hydrocarbon group that R^{e5}, R^{e6}, R^{e7} and R^{e8} represent include an alkyl group such as a methyl group, an ethyl group, a propyl group, a butyl group, a pentyl group, a hexyl group, a heptyl group, an octyl group, a decyl group, a dodecyl group, a tridecyl group, a tetradecyl group, a pentadecyl group, a hexadecyl group, a heptadecyl group and a stearyl group; an alkenyl group; and an alkynyl group. These aliphatic hydrocarbon groups can be linear or branched.

Among the atom or the group that R^{e5}, R^{e6}, R^{e7} and R^{e8} represent, R^{e5} and Res are preferably a hydrogen atom, and R^{c7} and R^{c8} are preferably a phenyl group.

The carbon number of the alkylene group that X^{e2} in the general formula (e-2) represents is preferably 1 to 10, more preferably 2 to 8, even more preferably 3 to 5.

Examples of the alkylene group that X^{e2} represents include a methylene group, an ethylene group, a 1,3-trimethylene group, a 1,4-tetramethylene group, a 1,5-pentamethylene group, and a 1,6-hexamethylene group.

The carbon number of the alkylidene group that X^{e2} represents is preferably 3 to 10, more preferably 3 to 8, even more preferably 3 to 5.

Examples of the alkylidene group that X^{e2} represents include an ethylidene group, a propylidene group, an isopropylidene group, a butylidene group, an isobutylidene group, a pentylidene group, and an isopentylidene group. Among these, preferred is a propylidene group.

In the case where the resin composition of the present embodiment contains the component (E), the content thereof is, though not specifically limited but from the viewpoint of compatibility, dielectric characteristics and heat resistance, preferably 0.01 to 10 parts by mass relative to 100 parts by mass of the sum total of the resin component in the resin composition, more preferably 0.1 to 6 parts by mass, even more preferably 0.5 to 4 parts by mass, especially preferably to 2 parts by mass.

### <Inorganic Filler (F)>

Containing an inorganic filler (F) [hereinafter this may be referred to as “component (F)”], the resin composition of the present embodiment tends to be more improved in point of the thermal expansion coefficient, the elasticity, the heat resistance and the flame retardance.

One kind alone of inorganic filler (F) or two or more kinds thereof can be used either singly or as combined.

Though not specifically limited, examples of the component (F) include silica, alumina, titanium oxide, mica, beryllia, barium titanate, potassium titanate, strontium titanate, calcium titanate, aluminum carbonate, magnesium hydroxide, aluminum hydroxide, aluminum silicate, calcium carbonate, calcium silicate, magnesium silicate, silicon nitride, boron nitride, clay, talc, aluminum borate and silicon carbide. Among these, from the viewpoint of thermal expansion coefficient, elasticity, heat resistance and flame retardance, preferred are silica, alumina, mica and talc, and more preferred are silica, alumina, and silica is even more preferred.

Examples of silica include precipitated silica having a high water content produced in a wet process, and a dry process silica not almost containing bound water and produced in a dry process. Further, the dry process silica is grouped into, for example, crushed silica, fumed silica and fused silica, depending on the difference in the production process.

Though not specifically limited, the particle size of the inorganic filler (F) is preferably 0.01 to 20 μm, more preferably 0.1 to 10 μm, even more preferably 0.2 to 1 μm, especially preferably 0.3 to 0.8 μm. Here, the particle size of the inorganic filler (F) indicates an average particle size, and is a particle diameter at the point corresponding to a volume 50%, on a cumulative frequency distribution curve of particle size where the total volume of the particles is 100%. The particle size of the inorganic filler (F) can be measured with a particle size distribution measuring device using a laser diffraction scattering method.

The shape of the inorganic filler (F) includes spheres and crushed granules, and spheres are preferred.

In the case where the resin composition of the present invention contains the inorganic filler (F), the content of the inorganic filler (F) in the resin composition is, though not specifically limited but from the viewpoint of low thermal expansion property, elasticity, heat resistance and flame retardance, preferably 10 to 70% by mass relative to the total solid content (100% by mass) of the resin composition, more preferably 20 to 65% by mass, even more preferably 30 to 60% by mass, especially preferably 40 to 55% by mass.

In the case where the resin composition of the present invention contains the inorganic filler (F), as needed and for the purpose of improving the dispersibility of the inorganic filler (F) and the adhesiveness thereof to organic components, a coupling agent can be used in the resin composition.

### <Flame Retardant (G)>

Containing a flame retardant (G), the resin composition of the present invention tends to have more improved flame retardancy.

One kind alone of flame retardant (G) or two or more kinds thereof can be used either singly or as combined. Further as needed, the resin composition can contain a flame retardant promoter.

Examples of the flame retardant (G) include a phosphorus-based flame retardant, a metal hydrate, and a halogen-based flame retardant. Among these, from the viewpoint of environmental problem, preferred are a phosphorus-based flame retardant and a metal hydrate.

### —Phosphorus-based Flame Retardant—

As the flame retardant, among those generally used as a flame retardant, any and every one containing a phosphorus atom can be used with no specific limitation, but from the viewpoint of environmental problem, those not containing a halogen atom are preferred.

The phosphorus-based flame retardant can be an inorganic phosphorus-based flame retardant, but from the viewpoint of dielectric characteristics, adhesiveness to conductors, heat resistance, glass transition temperature, low thermal expansion property and flame retardancy, preferred is an organic phosphorus-based flame retardant.

Examples of the inorganic phosphorus-based flame retardant include red phosphorus; ammonium phosphates such as monoammonium phosphate, diammonium phosphate, triammonium phosphate, and ammonium polyphosphate; inorganic nitrogen-containing phosphorus compounds such as phosphoric acid amides; phosphoric acid; and phosphine oxide.

Examples of the organic phosphorus-based flame retardant include an aromatic phosphate, a mono-substituted phosphinic acid diester, a di-substituted phosphinic acid ester, a metal salt of a di-substituted phosphinic acid, an organic nitrogen-containing phosphorus compound, a cyclic organic phosphorus compound, and a phosphine oxide compound. Among these, preferred are an aromatic phosphate, a metal salt of a di-substituted phosphinic acid, and a phosphine oxide compound. Here, example of the metal salt of a di-substituted phosphinic acid include lithium salts, sodium salts, potassium salts, calcium salts, magnesium salts, aluminum salts, titanium salts, and zinc salts. Among these, preferred are aluminum salts.

Examples of the aromatic phosphate include triphenyl phosphate, tricresyl phosphate, trixylenyl phosphate, cresyldiphenyl phosphate, cresyl-2,6-xylenyl phosphate, resorcinol bis(diphenyl phosphate), 1,3-phenylenebis(cli-2,6-xylenyl phosphate), bisphenol A-bis(diphenyl phosphate), and 1,3-phenylenebis(diphenyl phosphate).

Examples of the mono-substituted phosphonic acid diester include divinyl phenylphosphonate, diallyl phenylphosphonate, and bis(1-butenyl) phenylphosphonate.

Examples of the disubstituted phosphinic acid ester include phenyl diphenylphosphinate, and methyl diphenylphosphinate.

Examples of the metal salt of a di-substituted phosphinic acid include metal salts of a dialkylphosphinic acid, metal salts of a diallylphosphinic acid, metal salts of a divinylphosphinic acid, and metal salts of a diarylphosphinic acid. As these metal salts, preferred are aluminum salts.

Examples of the organic nitrogen-containing phosphorus compound include a phosphazene compound such as bis(2-allylphenoxy)phosphazene, and dicresylphosphazene; a melamine phosphate; a melamine pyrophosphate; a melamine polyphosphate, and a melam polyphosphate.

Examples of the cyclic organic phosphorus compound include 9,10-dihydro-9-oxa-10-phosphaphenanthrene-10-oxide, and 10-(2,5-dihydroxyphenyl)-9,10-dihydro-9-oxa-10-phosphaphenanthrene-10-oxide.

Examples of the phosphine oxide compound include paraxylylenebisdiphenylphosphine oxide, paraphenylenebisdiphenylphosphine oxide, ethylenebisdiphenylphoshine oxide, biphenylenebisdiphenylphosphine oxide, and naphthylenebisdiphenylphosphine oxide.

Among the above-mentioned organic phosphorus-based flame retardants, preferred are an aromatic phosphate, a metal salt of a di-substituted phosphinic acid, and a phosphine oxide compound, and more preferred are 1,3-phenylenebis(di-2,6-xylenyl phosphate), aluminum salts of dialkyl phosphinic acid, and paraxylenylenebisdiphenylphosphine oxide.

### —Metal Hydrate—

Examples of the metal hydrate include aluminum hydroxide hydrate and magnesium hydroxide hydrate.

### —Halogen-Based Flame Retardant—

Examples of the halogen-based flame retardant include a chlorine-based flame retardant, and a bromine-based flame retardant. Examples of the chlorine-based flame retardant include paraffin chloride.

In the case where the resin composition of the present embodiment contains a phosphorus-based flame retardant as the component (G), the content of the phosphorus-based flame retardant in the resin composition is, though not specifically limited but from the viewpoint of flame retardancy, moldability and heat resistance, preferably 0.2 to 10 parts by mass in terms of phosphorus atom relative to 100 parts by mass of the sum total of the resin component in the resin composition, more preferably 0.3 to 7 parts by mass, even more preferably 0.5 to 5 parts by mass, especially preferably 1 to 3 parts by mass.

### <Other Component (H)>

The resin composition of the present invention can further contain other component (H) [hereinafter this may be referred to as “component (H)”] than the above-mentioned components. Examples of the other component (H) include, except the above-mentioned components, a thermosetting resin, a thermoplastic polymer, a curing accelerator, a flame retardant, an additive and an organic solvent.

Each one kind alone of the component (H) or two or more kinds thereof can be used either singly or as combined.

Examples of the curing accelerator as the component (H) include an acid catalyst such as p-toluenesulfonic acid; an amine compound such as triethylamine, pyridine and tributylamine; a tertiary amine compound; a quaternary ammonium compound; a phosphorus compound such as triphenyl phosphine; an organic peroxide such as dicumyl peroxide, 2,5-dimethyl-2,5-bis(t-butylperoxy)hexyne-3,2,5-dimethyl-2,5-bis(t-butylperoxy)hexane, t-butylperoxyisopropyl monocarbonate, and α,α′-bis(t-butylperoxy)diisopropylbenzene; and a carboxylate with manganese, cobalt or zinc.

Examples of the additive as the component (H) include an antioxidant, a thermal stabilizer, an antistatic agent, a UV absorbent, a pigment, a colorant and a lubricant.

The amount of the component (H) to be used is not specifically limited, and can be within a range not interfering with the advantageous effects of the present invention.

### (Organic Solvent)

The resin composition of the present embodiment can contain an organic solvent. Dilution with an organic solvent tends to facilitate the handleability of the resin composition of the present embodiment and also the production of prepreg to be mentioned below. The resin composition containing an organic solvent can be generally referred to as a resin varnish or varnish.

Examples of the organic solvent include an alcohol solvent such as ethanol, propanol, butanol, methyl cellosolve, butyl cellosolve, and propylene glycol monomethyl ether; a ketone solvent such as acetone, methyl ethyl ketone, methyl isobutyl ketone, and cyclohexanone; an ether solvent such as tetrahydrofuran; an aromatic hydrocarbon solvent such as toluene, xylene, and mesitylene; a nitrogen atom-containing solvent such as dimethylformamide, dimethylacetamide, and N-methylpyrrolidone; a sulfur atom-containing solvent such as dimethyl sulfoxide; and an ester solvent such as γ-butyrolactone.

Among these, from the viewpoint of solubility, preferred are an alcohol solvent, a ketone solvent, a nitrogen atom-containing solvent, and an aromatic hydrocarbon solvent, more preferred is an aromatic hydrocarbon solvent, and even more preferred is toluene.

In the case where the resin composition of the present embodiment contains an organic solvent, the solid concentration of the resin composition is, though not specifically limited, preferably 30 to 90% by mass, more preferably 35 to 80% by mass, even more preferably 40 to 60% by mass. When the solid concentration of the resin composition falls within the above range, the handleability of the resin composition can be facilitated, and infiltration into substrates and appearance of the prepreg to be produced tend to be better. In addition, the solid concentration of the resin in prepreg to be mentioned below can be more readily adjusted, and prepreg having a desired thickness tends to be produced more easily.

### <Production Method for Resin Composition>

The resin composition of the present embodiment can be produced by mixing the above-mentioned component in a known manner. At that time, the components can be dissolved or dispersed with stirring. The conditions such as the mixing order, the temperature and the time are not specifically limited, and can be arbitrarily set depending on the kind of the raw materials.

### <Physical Properties of Resin Composition>

### (Dielectric Characteristics)

The permittivity (Dk) at 10 GHz of a cured product of the resin composition of the present embodiment is, though not specifically limited, preferably 3.0 or less, more preferably 2.9 or less, even more preferably 2.8 or less. The permittivity (Dk) is preferably smaller, and the lower limit thereof can be, though not specifically limited but in consideration of balance with other physical properties, for example, 2.3 or more, or can be 2.4 or more, or can be 2.5 or more. The permittivity (Dk) is a value complying with a cavity resonator perturbation method and is, more precisely, a value measured according to the method described in Examples. In the present specification, a mere expression of permittivity means a relative permittivity.

The dielectric loss tangent (DO at 10 GHz of a cured product of the resin composition of the present embodiment is, though not specifically limited, preferably 0.0050 or less, more preferably 0.0040 or less, even more preferably 0.0030 or less, especially preferably 0.0025 or less, and is most preferably 0.0022 or less. The dielectric loss tangent (Df) is preferably smaller, and the lower limit thereof can be, though not specifically limited but in consideration of balance with other physical properties, for example, 0.0010 or more, or can be 0.0013 or more, or can be 0.0015 or more. The dielectric loss tangent (Df) is a value complying with a cavity resonator perturbation method and is, more precisely, a value measured according to the method described in Examples.

### (Glass Transition Temperature)

Though not specifically limited, the glass transition temperature of the resin composition of the present embodiment, as measured according to the method described in Examples, is preferably 190° C. or higher, more preferably 200° C. or higher, even more preferably 210° C. or higher. The glass transition temperature is preferably higher, and can be, from the viewpoint of ease of production, 300° C. or lower, or can be 270° C. or lower, or can be 250° C. or lower.

### [Prepreg]

The prepreg of the present embodiment is a prepreg containing the resin composition of the present invention.

As the sheet-like fiber-reinforced substrate that the prepreg of the present embodiment contains, usable is any known sheet-like fiber-reinforced substrate used in various laminated plates for electric insulating materials.

Examples of the material for the sheet-like fiber-reinforced substrate include inorganic fibers of E glass, D glass, S glass or Q glass; organic fibers of polyimide, polyester, or tetrafluoroethylene; and mixtures thereof. For example, these sheet-like fiber-reinforced substrates have a shape of woven fabric, nonwoven fabric, robing, chopped strand mat or surfacing mat.

Though not specifically limited, the thickness of the sheet-like fiber-reinforced substrate is, for example, 0.02 to 0.5 mm.

From the viewpoint of resin composition infiltrability, and heat resistance, moisture absorption resistance and workability of laminated plates produced, the sheet-like fiber-reinforced substrate can be surface-treated with a coupling agent, or can be subjected to mechanical opening treatment.

The prepreg of the present embodiment can be produced, for example, by impregnating the sheet-like fiber-reinforced substrate with the resin composition of the present embodiment or by applying the resin composition to the substrate, and then optionally drying the resultant substrate.

As the method of impregnating the sheet-like fiber-reinforced substrate with the resin composition or applying the resin composition to the substrate, for example, a hot melt method or a solvent method can be employed.

A hot melt method is a method of impregnating the sheet-like fiber-reinforced substrate with the resin composition not containing an organic solvent, or applying the resin composition to the substrate. In one embodiment of the hot melt method, the resin composition is once applied to a releasable coating sheet, and the thus-applied resin composition is laminated on the sheet-like fiber-reinforced substrate. In another embodiment of the hot melt method, the resin composition is directly applied to the sheet-like fiber-reinforced substrate with a die coater.

A solvent method is a method of impregnating the sheet-like fiber-reinforced substrate with the resin composition containing an organic solvent, or applying the resin composition to the substrate. Specifically, for example, in one method, the sheet-like fiber-reinforced substrate is impregnated with the resin composition containing an organic solvent, and then dried.

The drying condition for the solvent method can be, for example, heating at 80 to 200° C. for 1 to 30 minutes. By drying, the organic solvent can be removed and the resin composition can be semi-cured (to be in a B-stage) to give the prepreg of the present embodiment.

The solid concentration derived from the resin composition in the prepreg of the present embodiment is, though not specifically limited, preferably 30 to 90% by mass. When the solid concentration derived from the resin composition in the prepreg falls within the above range, better moldability can be attained in producing laminated plates.

### [Resin Film]

The resin film of the present embodiment is a resin film containing the resin composition of the present embodiment.

The resin film of the present embodiment can be produced, for example, by applying the resin composition containing an organic solvent, that is, a resin varnish to a support and then heating and drying the support.

Examples of the support include a film of a polyolefin such as polyethylene, polypropylene or polyvinyl chloride; a film of a polyester such as polyethylene terephthalate [hereinafter this may be referred to as “PET”], or polyethylene naphthalate; various plastic films such as polycarbonate film or polyimide film; a metal foil such as a copper foil and an aluminum foil; and release paper.

The support can be surface-treated by mat treatment or corona treatment. The support can be release-treated with a silicone resin-based release agent, an alkyd resin-based release agent or a fluorine resin-based release agent.

Though not specifically limited, the thickness of the support is, for example, preferably 10 to 150 μm, more preferably 20 to 100 μm, even more preferably 25 to 50 μm.

As the coating device for coating with the resin varnish, a coating machine known to anyone skilled in the art can be used, and examples thereof include a comma coater, a bar coater, a kiss coater, a roll coater, a gravure coater, and a die coater. Depending on the intended film thickness, these coating devices can be arbitrarily selected.

The drying condition after coated with the resin composition can be selected in accordance with the content and the boiling point of the organic solvent, and is not specifically limited. For example, in the case of a resin varnish containing 40 to 60% by mass of an aromatic hydrocarbon solvent, a resin film can be favorably formed by drying at 50 to 200° C. for 3 to 10 minutes or so.

### [Laminated Plate]

The laminated plate of the present embodiment is a laminated plate containing the prepreg of the present embodiment and a metal foil. The metal foil-having laminated plate may be referred to as a metal-clad laminated plate.

The metal of the metal foil is not specifically limited so far as it is used for electric insulating materials, but from the viewpoint of electroconductivity, preferred are copper, gold, silver, nickel, platinum, molybdenum, ruthenium, aluminum, tungsten, iron, titanium, chromium, and alloys containing at least one metal element of these, more preferred are copper and aluminum, and even more preferred is copper.

The laminated plate of the present embodiment can be produced, for example, by arranging a metal foil on one surface or both surfaces of the prepreg of the present embodiment and hot press-molding it. In that case, one sheet alone of prepreg or two or more sheets thereof can be used either singly or as laminated.

Though not specifically limited, the condition for hot-press molding can be, for example, a temperature of 100 to 300° C., a pressure of 0.2 to 10 MPa, and a time of 0.1 to 5 hours. For hot-press molding, employable is a method of using a vacuum press and keeping a vacuum state for 0.5 to 5 hours.

### [Multilayer Printed Wiring Board]

The multilayer printed wiring board of the present embodiment contains at least one selected from the prepreg, the resin film and the laminated plate of the present embodiment.

Specifically, the multilayer printed wiring board of the present embodiment includes at least a multilayer structure that contains a cured product of the prepreg of the present embodiment, a cured product of the resin film of the present embodiment or the laminated plate of the present embodiment, and a conductor circuit layer.

The multilayer printed wiring board of the present embodiment can be produced by applying conductor circuit formation and multilayer-forming adhesion treatment to at least one selected from the group consisting of the prepreg, the resin film and the laminated plate of the present embodiment, according to a known method.

Conductor circuits can be formed, for example, by appropriately carrying out hole formation, metal plating and metal foil etching.

### [Semiconductor Package]

The semiconductor package of the present embodiment is a semiconductor package formed using the multilayer printed wiring board of the present embodiment.

The semiconductor package of the present embodiment is formed, for example, by mounting semiconductors on the multilayer printed wiring board of the present embodiment. The semiconductor package of the present embodiment can be produced, for example, by mounting semiconductor chips and memories on the multilayer printed wiring board of the present embodiment, according to a known method

The present embodiment is described above, but these are for exemplification for explaining the present invention, and are not intended to restrict the scope of the present invention only to these embodiments. Within a range not overstepping the scope and the spirit thereof, the present invention can be carried out in various modes different from the above-mentioned embodiments.

### EXAMPLES

Hereinunder, the present embodiment is described specifically with reference to the following Examples. However, the present embodiment is not limited to those Examples.

In each Example, the number-average molecular weight was measured according to the following process.

### (Measurement Method for Number-Average Molecular Weight)

The number average molecular weight was calculated from the calibration curve using a standard polyethylene, according to gel permeation chromatography (GPC). The calibration curve was approximated by a tertiary equation using standard polystyrene: TSK standard POLYSTYRENE (Type: A-2500, A-5000, F-1, F-2, F-4, F-10, F-20, F-40) [trade names by Tosoh Corporation). GPC measurement conditions are shown below.

Apparatus: High-performance GPC apparatus HLC-8320GPC

Detector: UV absorption detector UV-8320 [by Tosoh Corporation]

Column: Guardcolumn; TSK Guardcolumn Super HZ-L+ column; TSKgel Super HZM-N+TSKgel Super HZM-M+TSKgel Super H-RC (trade names, all by Tosoh Corporation)

Column size: 4.6×20 mm (guardcolumn), 4.6×150 mm (column), 6.0×150 mm (reference column)

Eluent: tetrahydrofuran

Sample concentration: 10 mg/5 mL

Injected amount: 25 μL

Flow rate: 1.00 mL/min

Measurement temperature: 40° C.

### (Measurement of Vinyl Group Modification Ratio)

In Production Example 2 to be mentioned below, the solution containing the component (c1) and the component (c2) before the start of reaction and the solution after the reaction were analyzed by GPC according to the above-mentioned method to determine the peak area derived from the component (c2) before and after the reaction. Next, according to the following formula, the vinyl group modification ratio of the component (c2) was calculated. The vinyl group modification ratio corresponds to the reduction ratio of the peak area derived from the component (c2) owing to reaction.

Vinyl Group Modification Ratio=[(peak area derived from the component (c2) before reaction)−(peak area derived from the component (c2) after reaction)]×100/(peak area derived from the component (c2) before reaction)

### Production Example 1: Production of Polyphenylene Ether Derivative

Toluene, one mol of polyphenylene ether “Zylon (registered trademark) S203A” (trade name by Asahi Kasei Corporation, number-average molecular weight=12,000−hereinunder this may be referred to as “raw material PPE”), and an allyl group-containing compound represented by the following general formula (1) in such an amount that the hydroxy group equivalent thereof relative to the raw material PPE could be 6 were put into a heatable and coolable, 2-L glass flask container equipped with a thermometer, a reflux condenser tube and a stirring device. Next, with stirring at 90 to 100° C., the components were dissolved. The amount of toluene used was such that the reaction concentration could be 35% by weight.

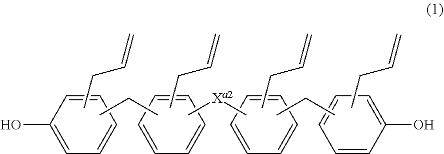

In the formula, X^{a2} is a divalent organic group, and is described in the same manner as that for X^{a2} in the general formula (a-5).

After dissolution of the allyl group-containing compound was visually confirmed, t-butylperoxyisopropyl monocarbonate in an amount of 2 parts by mass relative to 100 parts by mass of the raw material PPE and manganese octylate in an amount of 1.11 parts by mass relative to 100 parts by mass of the raw material PPE were added. Subsequently, this was reacted for redistribution at a solution temperature of 90 to 100° C. for 6 hours, and then cooled to 40° C. to give a polyphenylene ether derivative having an allyl group at the molecule terminal. A small amount of the reaction solution was taken out, and analyzed by GPC (in terms of polystyrene, eluent: tetrahydrofuran). As a result, it was confirmed that the double peak derived from the allyl group-containing compound changed to a single peak. The number-average molecular weight of the polyphenylene ether derivative was 4,200.

### Production Example 2: Production of Modified Conjugated Diene Polymer

33.5 parts by mass of 1,2-polybutadiene homopolymer (number-average molecular weight=1,200, vinyl group content=85% or more) as the component (c1), 1.47 parts by mass of 3,3′-dimethyl-5,5′-diethyl-4,4′-diphenylmethanebismaleimide as the component (c2), and a reaction catalyst and an organic solvent were put into a heatable and coolable, 2-L glass flask container equipped with a thermometer, a reflux condenser tube and a stirring device. Next, in a nitrogen atmosphere, this was stirred at 90 to 100° C. for 5 hours to react the component (c1) and the component (c2), thereby giving a solution of a modified conjugated diene polymer having a solid concentration of 35% by mass. The vinyl group modification ratio of the component (c2) was 40%, and the number-average molecular weight of the resultant modified conjugated diene polymer was 3,500.

### [Production of Resin Composition]

### Examples 1 to 5, Comparative Example 1

The components shown in Table 1 were blended according to the blending ratio shown in Table 1, and then stirred and mixed at room temperature or under heat at 50 to 80° C. to prepare a resin composition having a concentration of about 50% by mass. In Table 1, the unit of the blending amount of each component is part by mass, and in the case of a solution, it means part by mass in terms of the solid content therein. In Table 1, the parenthesized numerical value means the content of the phosphorus atom derived from each component in the resin composition.

[Production of Resin Film and Resin Plate Double-Clad with Copper Foil]

The resin composition obtained in each Example was applied to a PET film (trade name: G2-38, by Teijin Limited) having a thickness of 38 μm and then dried by heating at 170° C. for 5 minutes to produce a resin film in a B-stage condition.

The resin film was peeled from the PET film, and ground to give a B-stage resin powder, and this was put into a Teflon (registered trademark) sheet die-cut to a size of 1 mm thickness×50 mm length×35 mm width. Next, a low-profile copper foil (trade name: 3EC-VLP-18, by Mitsui Mining & Smelting Co., Ltd.) having a thickness of 18 μm was arranged above and below the resin powder-packed Teflon (registered trademark) sheet to prepare a laminated product before hot-press molding. The low-profile copper foil was so arranged that the M surface thereof faced the resin powder. Subsequently, the laminated product was hot-press molded under the condition of a temperature of 230° C., a pressure of 2.0 MPa, and a time of 120 minutes to mold and cure the resin powder to be a resin plate, thereby producing a resin plate double-clad with a copper foil. The thickness of the resin plate part of the resultant resin plate double-clad with a copper foil was 1 mm.

### [Evaluation Method and Measurement Method]

The resin plate double-clad with a copper foil, produced in Examples and Comparative Examples, was analyzed and evaluated according to the following methods. The results are shown in Table 1.

### (1. Compatibility Evaluation Method)

### (1) Production of Test Piece

The resin plate double-clad with a copper foil, as produced in each example, was immersed in a copper etching liquid being a 10 mass % solution of ammonium persulfate (by Mitsubishi Gas Chemical Company, Inc.) to remove the copper foil to give a resin plate.

Next, the resin plate was embedded in a casting resin and cured at room temperature for 12 hours to give a cast product of the resin plate. The resultant cast product was cut using a precision cutter (trade name; Refine Saw Excel, by Refine Tec Ltd.) to form a cross section of the resin plate. Subsequently, the cross section was subjected to platinum vapor-deposition to give a test piece.

(2) Observation with Scanning Electron Microscope

The cross section of the test piece obtained in the above was observed with a scanning electron microscope (SEM) (trade name: JSM-6010PLUA/LA, by JEOL Ltd.) at an acceleration voltage of 15 kV and under the condition of a reflection electron mode with no tilt to take a reflection electron image.

The image for calculating the area ratio R_{w} of the non-separation part was taken in arbitrary three fields of view each at an observation magnification of 100 times or 200 times (100 times in Examples 1, 3 to 5 and Comparative Example 1, 200 times in Comparative Example 2).

The image for calculating the average domain size D_{L} of the separation part was taken in arbitrary 6 fields of view each at an observation magnification of 65 times.

In Example 4, for investigating the influence of the observation magnification, reflection electron images were additionally taken at an observation magnification of 200 times and 1,000 times.

(3) Observation with Scanning Electron Microscope

In the reflected electronic image taken in the above, the phase-separated resin region looking relatively dark was specified as “separation part”, and the other resin region was specified as “non-separation part”. The separation part can be clearly discriminated as shown in FIG. 1.

### (4) Reflected Electronic Image Binarization

The reflected electronic image obtained in the above was binarized using an image processing analyzer software (trade name: Image-Pro Analyzer 7.0J, by Nippon Roper K.K.). The binarization condition is such that the threshold value of RGB is controlled within a range of 40 to 100, that the separation part specified in the above (3) can take one value while the non-separation part can take the other value in binarization to take a binarized image.

### (5) Calculation of Area Ratio R_{w} of Non-Separation Part

The area ratio of the region of the non-separation part of the resultant binarized image to the total region of the binarized image (area of the region of the non-separation part×100/area of the total region of the binarized image) was calculated, and a value was calculated by averaging the data of the area ratios on the three fields of view to be an area ratio R_{w} of the non-separation part. A larger value of the calculated R_{w} indicates more excellent compatibility.

### (6) Calculation of Average Domain Size D_{L} of Separation Part

The average domain size D_{L} of the separation part was determined by averaging the domain size values of the domains having a size ranging from the 2nd to the 6th among the domains having a larger domain size counted from the larger domain size among the domains in the separation part in the reflected electronic image of the 6 fields of view taken in the above. A smaller value of the average domain size D_{L} of the separation part thus calculated indicates more excellent compatibility. The domain size was measured according to the method mentioned above.

### (2. Measurement Method for Permittivity and Dielectric Loss Tangent

The resin plate double-clad with a copper foil, as produced in each example, was immersed in a copper etching liquid being a 10 mass % solution of ammonium persulfate (by Mitsubishi Gas Chemical Company, Inc.) to remove the copper foil to give a test piece of 2 mm×50 mm. Next, according to a cavity resonator perturbation method, the relative permittivity (Dk) and the dielectric loss tangent (Df) of the test piece were measured at an ambient temperature of 25° C. and in a 10 GHz zone.

### (3. Measurement Method for Glass Transition Temperature)

The copper foil on both sides of the resin plate double-clad with a copper foil obtained in each example as etched away to give a test piece of 5 mm square. Next, using a thermal mechanical analyzer (TMA) [by TA Instruments Japan, Q400 (Model Number)], the glass transition temperature of the test piece was measured according to the IPC (The Institute for Interconnecting and Packaging Electronic Circuits) standard.

TABLE 1

| | | | | | | | | Compar- |
|---|---|---|---|---|---|---|---|---|
| | | | | | | | | ative |
| | | | Example | | | | | Example |
| | | | 1 | 2 | 3 | 4 | 5 | 1 |
| Blending | Component (A) | polyphenylene ether derivative | 1.2 | 1.2 | 1.2 | 1.2 | 1.2 | 1.2 |
| Amount of | Component (B) | maleimide compound (B-1) | 21.1 | 21.1 | 21.1 | 21.1 | 21.1 | 21.1 |
| Compounds | | maleimide compound (B-2) | 2.3 | | | | | |
| | Component (C) | unmodified conjugated diene polymer | | 8.2 | | | | |
| | | modified conjugated diene polymer | 8.2 | | 8.2 | 8.2 | 8.2 | 8.2 |
| | Component (D) | hydrogenated styrene-based | 13.0 | 13.0 | 13.0 | 13.0 | 13.0 | 13.0 |
| | | thermoplastic elastomer | | | | | | |
| | Component (E) | modified imidazole compound of | 0.5 | 1.0 | 3.0 | | | |
| | | general formula (e-1) | | | | | | |
| | | modified imidazole compound of | | | | 0.5 | | |
| | | general formula (e-2) | | | | | | |
| | | 2-ethyl-4-methylimidazole | | | | | 0.5 | |
| | Component (F) | silica | 47.4 | 47.4 | 47.4 | 47.4 | 47.4 | 47.4 |
| | Component (G) | 1,3-phenylenebis(di-2,6-xylenyl phosphate) | (0.5) | (0.5) | (0.5) | (0.5) | (0.5) | (0.5) |
| | | phosphoric acid metal salt | (0.5) | (1.0) | (1.0) | (0.5) | (0.5) | (0.5) |
| | | paraxylylenebisdiphenylphosphine oxide | (0.5) | (0.5) | (0.5) | (0.5) | (0.5) | (0.5) |
| | Component (H) | α,α′-di(t- | 2.0 | 2.0 | 2.0 | 2.0 | 2.0 | 2.0 |
| | | butylperoxy)diisopropylbenzene | | | | | | |
| Evaluation | Compatibility | Area Ratio R_{w} (%) of | 72 | 84 | 91 | 64 | 58 | 59 |
| Results | | non-separation part | | | | | | |
| | | (observation magnification 100 to 200 times) | | | | | | |
| | | Average Domain Size D_{L} (μm) of | 39 | 48 | 32 | 50 | 80 | 158 |
| | | separation part | | | | | | |
| | | (observation magnification 65 times) | | | | | | |
| | Dielectric | Permittivity (Dk) (10 GHz) | 2.77 | 2.79 | 2.78 | 2.80 | 2.76 | 2.79 |
| | Characteristics | Dielectric Loss Tangent (Df) (10 GHz) | 0.0020 | 0.0024 | 0.0021 | 0.0025 | 0.0023 | 0.0031 |
| | Heat Resistance | Glass Transition Temperature (° C.) | 211 | 205 | 226 | 223 | 215 | 212 |

* In the Table, parenthesized numerical data mean the content of the phosphorus atom derived from each component in the resin composition.

Details of the components shown in Table 1 are as follows.

### [Component (A)]

Polyphenylene ether derivative: Polyphenylene ether derivative produced in Production Example 1.

### [Component (B)]

Maleimide compound (B-1): Biphenylaralkyl-type maleimide (trade name: “MIR-3000”, by Nippon Kayaku Co., Ltd.).

Maleimide compound (B-2): 3,3′-dimethyl-5,5′-diethyl-4,4′-diphenylmethanebismaleimide.

### [Component (C)]

Unmodified conjugated diene polymer: 1,2-polybutadiene homopolymer (number-average molecular weight=1,200, vinyl group content=85% or more).

Modified conjugated diene polymer: modified conjugated diene polymer produced in Production Example 2.

### [Component (D)]

Hydrogenated styrene-based thermoplastic elastomer: styrene-ethylene-butylene-styrene (SEBS) copolymer (trade name “Krayton (registered trademark) MD1653”, by Krayton Polymer Japan, melt flow rate 5.0 g/10 min, styrene content 30%, hydrogenation ratio 100%).

### [Component (E)]

Modified imidazole compound represented by the general formula (e-1): compound of the general formula (e-1) where R^{e1}, and R^{e2} are methyl groups, Rea, and R^{e4} are ethyl groups, and X^{e1} is a 1,4-tetramethylene group.

Modified imidazole compound represented by the general formula (e-2): compound of the general formula (e-2) where R^{e5}, and R^{e6} are hydrogen atoms, R^{e7} and R^{e8} are phenyl groups, and X^{e2} is a propylidene group.

2-Ethyl-4-methylimidazole.

### [Component (F)]

Silica: spherical molten silica, average particle size=0.5 μm.

### [Component (G)]

1,3-Phenylenebis(cli-2,6-xylenyl phosphate): phosphorus content 9.0% by mass.

Phosphoric acid metal salt: aluminum dialkylphosphinate, disubstituted phosphinic acid metal salt, phosphorus content 23.5% by mass (trade name “OP-935”, by Clariant Corporation).

Paraxylylenebisdiphenylphosphine oxide: phosphorus content 12.0% by mass.

### [Component (E)]

α,α′-bis(t-butylperoxy)diisopropylbenzene.

As obvious from Table 1, it is known that the cured products obtained in Examples 1 to 5 of the present embodiment have an area ratio R_{w} of the non-separation part of 50% or more, and an average domain size D_{L} of the separation part of 120 μm or less, and are excellent in dielectric characteristics and heat resistance. On the other hand, the cured product of Comparative Example 1 whose average domain size D_{L} of the separation part is more than 120 μm was poor in dielectric characteristics.

In three fields of view at an observation magnification of 200 times, the cured product obtained in Example 4 was analyzed to calculate the area ratio R_{w} of the non-separation part. All the R_{w} values in the three fields of view fell within a range of ±1% of the average value. On the other hand, the same test pieces were analyzed to calculate the area ratio R_{w} of the non-separation part in three fields of view at an observation magnification of 1,000 times, and there existed a difference of at most 5.7% relative to the average value. From the results, it is known that the compatibility evaluation method of the first mode of the present embodiment realizes excellent reproducibility when the observation magnification is 50 to 250 times.

### INDUSTRIAL APPLICABILITY

The compatibility evaluation method for a thermosetting resin composition of the present embodiment enables evaluation of the compatibility that has an influence on the physical properties such as dielectric characteristics and heat resistance of a thermosetting resin composition, and is therefore useful for substrate materials for use in printed wiring boards.

### REFERENCE SIGNS LIST

1 Non-Separation Part
2 Separation Part

1. A method for evaluating a compatibility of a thermosetting resin composition containing at least two kinds of resins and an inorganic filler, the method comprising the following steps 1A and 2A:
Step 1A: a step of obtaining a reflected electronic image of a cross section of a cured product of the thermosetting resin composition using a scanning electron microscope at an observation magnification of 50 to 250 times; and
Step 2A: a step in which, in the reflected electronic image, a phase-separated resin region is referred to as a separation part and a remaining region is referred to as a non-separation part, and the image is binarized such that the separation part has one value and the non-separation part has the other value, and an area ratio of a region of the non-separation part of the resultant binarized image to a total region of the binarized image (the area of the region of the non-separation part×100/area of the total region of the binarized image) is calculated as the area ratio R_{w} of the non-separation part.

2. A method for evaluating a compatibility of a thermosetting resin composition containing at least two kinds of resins and an inorganic filler, the method comprising the following steps 1B and 2B:
Step 1B: a step of obtaining a reflected electronic image of a cross section of a cured product of the thermosetting resin composition using a scanning electron microscope; and
Step 2B: a step in which, in the reflected electronic image, a phase-separated resin region is referred to as a separation part and an average domain size D_{L} of the separation part is determined.

3. The compatibility evaluation method of a thermosetting resin composition according to claim 2, wherein the step 1B is a step of obtaining the reflected electronic image of the cross section of the cured product of the thermosetting resin composition using the scanning electron microscope at an observation magnification of 50 to 200 times.

4. A thermosetting resin composition comprising at least two kinds of resins and an inorganic filler, wherein:
the area ratio R_{w} of the non-separation part obtained with a scanning electron microscope under the condition of an observation magnification of 100 times or 200 times in the compatibility evaluation method according to claim 1 is 50% or more, and
in the compatibility evaluation method according to the claim 2, the condition of the observation magnification of the scanning electron microscope is 65 times, and the average domain size D_{L} of the separation part, as obtained by averaging the domain size values of the domains having a size ranging from the 2nd to the 6th among the domains having a larger domain size counted from the larger domain size among the domains in the separation part observed in at least three fields of view, is 12011m or less.

5. A prepreg comprising the thermosetting resin composition according to claim 4.

6. A resin film comprising the thermosetting resin composition according to claim 4.

7. A laminated plate comprising the prepreg according to claim 5 and a metal foil.

8. A multilayer printed wiring board comprising the laminated plate according to claim 7.

9. A semiconductor package formed using the multilayer printed wiring board according to claim 8.

10. A multilayer printed wiring board comprising the prepreg according to claim 5.

11. A semiconductor package formed using the multilayer printed wiring board according to claim 10.

12. A multilayer printed wiring board comprising the resin film according to claim 6.

13. A semiconductor package formed using the multilayer printed wiring board according to claim 12.
